(19) United States
(12) Patent Application Publication
(10) Pub. No.: US 2006/0275299 A1
(43) Pub. Date: Dec. 7, 2006

(54) Use of protein tyrosine phosphatase inhibitors for prevention and/or treatment of cancer

(76) Inventors: Rob Hooft Van Huijsduijnen, Bernex (CH); Sebastien Walchli, Oslo (NO)

Correspondence Address: FISH & NEAVE IP GROUP;ROPES & GRAY LLP, ONE INTERNATIONAL PLACE, BOSTON, MA, 02110-2624, US

(21) Appl. No.: 10/530,106

(22) PCT Filed: Sep. 29, 2003
(86) PCT No.: PCT/EP03/50666

§ 371 Date: Oct. 18, 2005

(30) Foreign Application Priority Data
Oct. 2, 2002 (EP) 02022227.9

(51) Int. Cl. A61K 39/395

(52) U.S. Cl. 424/146.1; 424/155.1

(57) ABSTRACT

The invention relates to the use of a protein tyrosine phosphatase inhibitor, such as a cross-linker of a protein tyrosine phosphatase (PTP), in particular Sap-1, in the preparation of a medicament for treatment and/or prevention of cancer, and to methods of treating and/or preventing cancer using these inhibitors.

### FIELD OF THE INVENTION

The present invention is in the field of cancer. More specifically, it relates to the use of an inhibitor, in particular an agent cross-linking a protein tyrosine phosphatase (PTP), for the treatment and/or prevention of cancer. A cross-linker for the receptor-like PTP Sap-1 is preferably used for treatment or prevention of gastrointestinal cancers.

### BACKGROUND OF THE INVENTION

Cancer is the second cause of death in the developed world. Successful treatment of cancer requires elimination of all cancer cells, whether at the primary site, i.e. local-regional areas, or metastatic to other regions of the body. The major modalities of therapy are surgery and radiotherapy (for local and local-regional disease) and chemotherapy (for systemic sites). Other important methods include endocrine therapy (for selected cancers, e.g., prostate, breast, endometrium, liver), immunotherapy (procedures aimed at boosting the patient's immune system against the tumor), and thermotherapy (cryotherapy and heat). Multimodality therapy combines the assets of each of these.

Tumors of the gastrointestinal tract are among the most prevalent cancers today. These tumors, also called gastrointestinal cancers, include esophageal tumors, stomach cancer, small-bowel tumors, large-bowel tumors, and pancreatic cancers.

Esophageal Tumors

Most primary esophageal cancers are carcinomas. The most common malignant tumor of the esophagus is epidermoid carcinoma, followed by adenocarcinoma. Other malignant tumors of the esophagus include lymphoma, leiomyosarcoma, and metastatic cancer.

Stomach Cancer

Stomach Cancer includes gastric adenocarcinoma, which accounts for 95% of malignant tumors of the stomach. Less common are lymphomas (which may be localized primarily in the stomach) and leiomyosarcomas. The incidence of stomach cancer varies worldwide; for example, it is extremely high in Japan, Chile, and Iceland. In Japan its incidence increases with age, with more than 75% of patients over 50 years.

Treatment of stomach cancer includes excision of the tumor. The prognosis is good if the tumor is limited to the mucosa and submucosa. Combined chemotherapy and radiotherapy may also be curative in gastric lymphoma. Gastric adenocarcinoma patients with malignant ulcers have the best results, possibly because symptoms bring them to the physician earlier. Chemotherapy may be palliative for patients with metastases; radiotherapy with chemotherapy may be indicated for patients with locally unresectable tumors, but results are generally disappointing. Adjuvant chemotherapy or combined chemotherapy and radiotherapy are investigational techniques used after gastric resection.

Surgery for cancer involves removal of most or all of the stomach and adjacent lymph nodes. Metastases or extensive tumors preclude this type of cure.

Small-Bowel Tumors

These include benign and malignant tumors. Jejunal and ileal tumors account for 1 to 5% of gastrointestinal tract tumors. Predominantly benign, they include leiomyomas, lipomas, neurofibromas, and fibromas; all may cause symptoms requiring surgery. Polyps are more common in the colon than in the small bowel. Vascular tumors in the small bowel are multicentric in 55% of the cases. Hereditary hemorrhagic telangiectasia (Rendu-Osler-Weber syndrome) is an inborn progressive tendency to form dilated endothelial spaces. Hemangiomas may bleed or intussuscept. Angiodysplasias or arteriovenous malformations, consequences of aging, tend to occur in the distal small bowel or cecum.

Malignant tumors include e.g. a denocarcinoma. Usually these arise in the proximal jejunum and cause minimal symptoms. In the presence of Crohn's disease, the tumors tend to occur distally and in bypassed or inflamed bowel loops. Adenocarcinoma occurs more often in Crohn's disease of the small bowel than in Crohn's disease of the colon. Primary malignant lymphoma arising in the ileum may produce a long, rigid segment. Small-bowel lymphomas arise frequently in celiac sprue. The small bowel, particularly the ileum, is the second most common site (after the appendix) of carcinoid tumors. Multiple tumors occur in 50% of cases. Of those >2 cm in diameter, 80% have metastasized locally or to the liver by the time of operation. About 30% of small-bowel carcinoids cause obstruction, pain, bleeding, or the carcinoid syndrome. Treatment is generally surgical resection, and repeat operations may be required.

Kaposi's sarcoma, first described as a disease of elderly Jewish and Italian men, occurs in an aggressive form in Africans, transplant recipients, and AIDS patients, who have gastrointestinal tract involvement 40 to 60% of the time. Lesions may occur anywhere in the gastrointestinal tract but usually are found in the stomach, small bowel, or distal colon. Gastrointestinal tract lesions usually are asymptomatic, but bleeding, diarrhea, protein-losing enteropathy, and intussusception may occur. A second primary intestinal malignancy occurs in ≦20% of patients; most often it is lymphocytic leukemia, non-Hodgkin's lymphoma, Hodgkin's disease, or adenocarcinoma of the gastrointestinal tract.

Large-Bowel Tumors

Among large-bowel tumors, polyps of the colon and rectum are common. A polyp (a clinical term without pathologic significance) is any mass of tissue that arises from the bowel wall and protrudes into the lumen. Incidence ranges from 7 to 50%; the higher figure includes very small polyps (usually hyperplastic polyps or adenomas) found at autopsy. Polyps are detected in about 5% of patients by routine barium enemas and more often by flexible fiberoptic sigmoidoscopy, colonoscopy, or air contrast barium enema. Polyps, often multiple, occur most commonly in the rectum and sigmoid and decrease in frequency toward the cecum. About 25% of patients with cancer of the large bowel also have satellite adenomatous polyps.

The cancer risk of a tubular adenoma is controversial, but strong evidence suggests that it can become malignant. Risk of malignancy is related to size; a 1.5-cm tubular adenoma presents a 2% risk. As its size increases, its glands become villous. When >50% of its glands are villous, it is called a villoglandular polyp; its malignancy potential is still that of a tubular adenoma. When >80% of the glands are villous, the polyp is called a villous adenoma, which becomes malignant in about 35% of cases. A villous adenoma presents a greater risk of malignancy than a tubular adenoma of the same size.

Polyps are treated by complete removal with a snare or electrosurgical biopsy forceps after total colonoscopy. Large villous adenomas have a high potential for malignancy and must be excised completely.

Treatment of a cancerous polyp depends on the depth of invasion of the anaplastic epithelium into the polyp's stalk, the proximity of the endoscopic line of resection, and the degree of differentiation of the malignant tissue. If anaplastic epithelium is confined above the muscularis mucosa, if the line of resection in the polyp's stalk is clear, or if the lesion is well differentiated, endoscopic excision and close endoscopic follow-up should suffice. Invasion through the muscularis mucosa provides access to lymphatics and increases the potential for lymph node metastasis. The excision of polyps without a clear resection line or poorly differentiated lesions should be followed by segmental resection of the colon.

Juvenile polyps are usually non-neoplastic, often outgrow their blood supply, and autoamputate at puberty. Treatment is required only for uncontrollable bleeding or intussusception. Hyperplastic polyps, also non-neoplastic, are common in the colon and rectum. Inflammatory polyps and pseudopolyps occur in chronic ulcerative colitis and in Crohn's disease of the colon.

Colorectal Cancer

In Western countries, the colon and rectum account for more new cases of cancer per year than any other anatomic site except the lung. Colorectal cancer is the most frequent cause of death among visceral malignancies that affect both sexes. Its incidence begins to rise at age 40 and peaks at age 60 to 75 years. Cancer of the colon is more common in women; cancer of the rectum is more common in men. Synchronous cancers (more than one) occur in 5% of patients.

At least four genes located on chromosomes 2, 3, and 7 have been shown to be mutated in some cases of the Lynch syndrome. Other predisposing factors include chronic ulcerative colitis, granulomatous colitis, and familial polyposis (which includes Gardner's syndrome); in these disorders, the risk of cancer at any time is related to the age of onset and duration of the underlying disease.

Populations with a high incidence of colorectal cancer eat low-fiber diets that are high in animal protein, fat, and refined carbohydrates. Carcinogens may be ingested in the diet but are more likely to be produced from dietary substances or from biliary or intestinal secretions, probably by bacterial action. The exact mechanism is unknown.

Colorectal cancer spreads by direct extension through the bowel wall, hematogenous metastasis, regional lymph node metastasis, perineural spread, and intraluminal metastasis.

Adenocarcinoma of the colon and rectum grows slowly, and a long interval elapses before it is large enough to produce symptoms. Early diagnosis depends on routine examination. Symptoms depend on the lesion's location, type, extent, and complications. The right colon has a large caliber and a thin wall. Because the colon contents are liquid, obstruction is a late event. Tumors, which are usually fungating, may grow large and be palpable through the abdominal wall. Bleeding is usually occult. Fatigue and weakness caused by severe anemia may be the only complaints. The left colon has a smaller lumen, the feces are semisolid, and cancer tends to encircle the bowel, causing alternating constipation and increased stool frequency or diarrhea. Partial obstruction with colicky abdominal pain or complete obstruction may be the presenting picture. The stool may be streaked or mixed with blood. In cancer of the rectum, the most common presenting symptom is bleeding with defecation. Whenever rectal bleeding occurs, even with obvious hemorrhoids or known diverticular disease, coexisting cancer must be ruled out. Tenesmus or a sensation of incomplete evacuation may be present. Pain is noticeably absent until perirectal tissue is involved.

Primary treatment consists of wide surgical resection of the colon cancer and regional lymphatic drainage after the bowel is prepared. Surgical cure is possible in 70% of patients. The best 5-year survival rate for cancer limited to the mucosa approaches 90%; with penetration of the muscularis propria, 80%; with positive lymph nodes, 30%. When the patient is an unacceptable surgical risk, some tumors can be controlled locally by electrocoagulation. Preliminary results from studies of adjuvant radiotherapy after curative surgery of rectal (but not colon) cancer suggest that local tumor growth can be controlled, recurrence delayed, and survival improved in patients with limited lymph node involvement.

Rectal cancer patients with one to four positive lymph nodes derive most benefit from combined radiotherapy and chemotherapy; when more than four positive lymph nodes are found in the resected specimen, combined modalities are less effective. The effective regimen studied is fluorouracil (5-FU) with or without folinic acid. Careful planning by radiation physicists, with special attention to avoid small-bowel injury, is necessary when such combined chemotherapy and radiotherapy regimens are used.

Use of pre-operative radiotherapy to improve the resectability rate of rectal cancer is controversial; experts disagree on whether this treatment increases operability or decreases the incidence of detection of regional lymph node metastases. Controlled studies are exploring the use of pre-operative vs. postoperative chemotherapy and radiotherapy in patients with rectal cancer.

Chemotherapy with 5-FU combined with levamisole or folinic acid has not proven as effective as surgical adjuvants in properly controlled trials of node-positive (stage III, Dukes' C) colon cancer.

The frequency of follow-up after curative surgery for colorectal cancer is controversial. Most authorities recommend two annual inspections of the remaining bowel with colonoscopy or x-rays and, if negative, repeat evaluations at 2- to 3-year intervals.

Anorectal Cancer

The most common anorectal cancer is adenocarcinoma. Other tumors include squamous cloacogenic carcinoma, melanoma, lymphoma, and various sarcomas. Epidermoid (nonkeratinizing squamous cell or basaloid) carcinoma of the anorectum accounts for 3 to 5% of distal large-bowel cancers. Chronic fistulas, irradiated anal skin, leukoplakia, lymphogranuloma venereum, Bowen's disease (intraepithelial carcinoma), and condyloma acuminatum are predisposing causes. A major association with infection with human papillomavirus has been demonstrated. Metastasis occurs along the lymphatics of the rectum and in the inguinal lymph nodes. Basal cell carcinoma, Bowen's disease (intradermal carcinoma), extramammary Paget's disease, cloacogenic carcinoma, and malignant melanoma are less common.

Pancreatic Cancer/Tumors

Exocrine tumors of the pancreas develop from ductal and acinar cells. Endocrine tumors arise from islet and gastrin-producing cells and often produce many hormones.

Exocrine Tumors

Ductal Adenocarcinoma arise from duct cells nine times more often than from acinar cells; 80% occur in the head of the gland. Adenocarcinomas appear at the mean age of 55 years and occur 1.5 to 2 times more often in men. Symptoms occur late in the course of disease; by diagnosis, 90% of patients have a tumor that is locally advanced and has directly involved retroperitoneal structures, spread to regional lymph nodes, or metastasized to the liver or lung. Weight loss and abdominal pain occur in most patients with advanced disease. Adenocarcinomas may produce obstructive jaundice and, if in the body and tail, splenic vein obstruction, splenomegaly, gastric and esophageal varices, and GI hemorrhage.

Neither single nor combinations of drugs prolong or enhance the quality of life. Single agents that have been tested are 5-FU, methotrexate, actinomycin D, doxorubicin, carmustine, semustine, and streptozocin. Combinations that have been tested include FAM (5-FU, doxorubicin, mitomycin C), FAMMC (5-FU, doxorubicin, mitomycin C, semustine), and SMF (streptozocin, mitomycin, 5-FU). In contrast, the combination of 5-FU and radiotherapy (4,000 to 5,000 cGy) improves survival compared with radiotherapy alone in patients with locally unresectable disease. Newer agents (e.g., gemcitabine) may be more effective than 5-FU-based chemotherapy. For patients with locally unresectable tumors, intraoperative electron beam radiotherapy (4,500 to 5,500 cGy) or ¹²⁵I implant (120 to 210 cGy) may limit tumor progression locally but does not improve survival compared with external beam radiotherapy. Most patients with locally inoperable tumor are offered chemotherapy and radiotherapy; those with hepatic metastasis are offered chemotherapy alone.

Cystadenocarcinoma is a rare adenomatous pancreatic cancer that arises as a malignant degeneration of a mucous cystadenoma and presents as upper abdominal pain and a palpable abdominal mass.

Intraductal Papillary-Mucinous Tumor is a recently described syndrome of dilatation of the main pancreatic duct or branch ducts, with mucin overproduction.

Endocrine Tumors

Pancreatic endocrine tumors have two general presentations. Non-functioning tumors may cause obstructive symptoms of the biliary tract or duodenum, bleeding into the gastrointestinal tract, or form abdominal masses. Functioning tumors hypersecrete a particular hormone, causing various syndromes, including hypoglycemia (insulinoma hypersecretes insulin); Zollinger-Ellison syndrome (gastrinoma hypersecretes gastrin); vipoma (vasoactive intestinal peptide or prostaglandins E and E₂ hypersecretion); carcinoid syndrome, diabetes (glucagonoma hypersecretes glucagon); Cushing's syndrome (ACTH hypersecretion); and mild hyperglycemia with cholelithiasis (somatostatinoma). These clinical syndromes also occur sometimes in multiple endocrine neoplasia, in which tumors or hyperplasia affects two or more endocrine glands, usually the parathyroid, pituitary, thyroid, or adrenals.

Insulinoma is a rare islet cell tumor that hypersecretes insulin. Insulinoma is a tumor of pancreatic beta-cells or, rarely, diffusely hyperplastic beta-cells. Of all insulinomas, 80% are single and may be curatively resected if identified. Only 10% of insulinomas are malignant. They develop in 1/250,000 persons at a median age of 50 years, except in multiple endocrine neoplasia type I (about 10% of insulinomas), where they develop in the third decade of life. Insulinomas associated with multiple endocrine neoplasia type I are more likely to be multiple.

Treatment comprises overall surgical cure.

Zollinger-Ellison Syndrome (Z-E Syndrome; Gastrinoma) is a syndrome characterized by marked hypergastrinemia, gastric hypersecretion, and peptic ulceration caused by a gastrin-producing tumor of the pancreas or the duodenal wall.

Sometimes the gastrinoma is in the splenic hilum, mesentery, stomach, lymph node, or ovary. Most patients have multiple tumors, of which 50% are malignant. Usually, the tumors are small (<1 cm in diameter) and grow and spread slowly. They occur predominantly in patients with other endocrine abnormalities, especially of the parathyroids and, less often, of the pituitary and adrenal glands.

Treatment includes administration of the H⁺, K⁺-ATPase inhibitor omeprazole, which markedly reduces gastric parietal cell H⁺ secretion. It alleviates symptoms, promotes ulcer healing, and is now the treatment of choice. The initial starting dosage is 60 mg/day po, but higher doses may be needed in 30% of patients, especially those with severe reflux esophagitis, previous gastric surgery, multiple endocrine neoplasia type I, or large or metastatic tumors. Patients need to take omeprazole indefinitely unless they undergo surgical cure, which is possible in 20% of patients with nonfamilial Z-E. Somatostatin analog may also decrease gastric acid production and may be palliative in patients not responding well to omeprazole.

Vipoma is a tumor of non-beta pancreatic islet cells causing a syndrome of watery diarrhea, hypokalemia, and achlorhydria. Of these tumors, 50 to 75% are malignant, and some may be quite large (7 cm) at diagnosis. The major clinical features are prolonged massive watery diarrhea (fasting stool volume >750 to 1,000 mL/day and nonfasting volumes of >3,000 muday) and symptoms of hypokalemia, acidosis, and dehydration. Of patients, 50% have relatively constant diarrhea, whereas the rest have alternating severe and moderate diarrhea; 33% have diarrhea <1 year before diagnosis, but 25% have diarrhea ≧5 years before diagnosis. Lethargy, muscular weakness, nausea, vomiting, and crampy abdominal pain are frequent. Hyperglycemia and impaired glucose tolerance occur in ≦50% of patients. Rarely, flushing similar to the carcinoid syndrome occurs during attacks of diarrhea.

Tumor resection is curative in 50% of patients with a localized tumor. In those with metastatic tumor, resection of all visible tumor may provide temporary relief of symptoms. The combination of streptozocin and doxorubicin may reduce diarrhea and tumor mass if objective response occurs (in 50 to 60%). Chemotherapy is not curative.

Glucagonoma is a pancreatic alpha-cell glucagon-secreting tumor that produces hyperglycemia. Glucagonomas are very rare but similar to other islet cell tumors in that the primary and metastatic lesions are slow-growing: Of glucagonomas, 80% are malignant.

Protein tyrosine kinases (PTKs) have been shown to be involved in the development of cancer. PTKs such as the kinases src, abl, fps, yes, fyn, lyn, lck, blk, hck, fgr and yrk (reviewed by Bolen et al., 1992) are involved in the cellular proliferative and metabolic signal transduction pathway and thus could be expected, and have been shown, to be involved in many PTK-mediated cancers. For example, mutated src (v-src) has been shown to be an oncoprotein (p60^{v-src}) in chicken. Moreover, its cellular homolog, the proto-oncogene p60^{c-src} transmits oncogenic signals of many receptors. Over-expression of E.G.F.R. (epidermal growth factor receptor) or HER2/neu in tumors leads to the constitutive activation of p60^{c-src}, which is characteristic of malignant cells but not observed in normal cells. On the other hand, mice deficient in the expression of c-src exhibit an osteopetrotic phenotype, indicating a key participation of c-src in osteoclast function and a possible involvement in related disorders.

The activity of p60^{c-src} oncogenic kinase is negatively regulated by Csk-mediated phosphorylation of its C-terminal Tyr₅₂₇, which then folds back onto the N-terminal SH2 domain, resulting in the suppression of kinase activity [(Liu et al. 1993) and citations therein].

With the growing recognition of the dynamic character of phosphorylation and dephosphorylation events in signaling (Espanel et al. 2001), efforts have been made to identify Protein Tyrosine Phosphatases (PTPs) that remove this autoinhibitory phosphate group. In cancers where c-src or related family members have not escaped control through mutation of this C-terminal tyrosine (Thomas and Brugge 1997), specific inhibitors for such a PTP could be of therapeutic use. A number of PTPs have been implicated in the activation src-family kinases, including PTP-α (Zheng et al. 1992; den Hertog et al. 1993; Bhandari et al. 1998), CD-45 (Cahir McFarland et al. 1993; Hurley et al. 1993), PTP-D1 (Moller et al. 1994), SHP-2 (Peng and Cartwright 1995), GLEPP-1 (Suhr et al. 2001) and PTP1B (Bjorge et al. 2000b; Cheng et al. 2001), but only few of these are known to be overexpressed in human tumors (Wiener et al. 1994; Bjorge et al. 2000b).

To date, five PTPs have been clearly associated with human cancer: PTP-DEP (Ruivenkamp et al. 2002) is frequently mutated in human tumors. Prl-3 is overexpressed in metastases (Saha et al. 2001), and cdc25 (Galaktionov et al. 1995) and Sap-1 (Matozaki et al. 1994; Seo et al. 1997), are overexpressed in various cancers.

Sap-1 (stomach cancer associated PTP) was cloned in 1994 as a new member of the type I transmembrane PTP family (Matozaki and Kasuga 1996). Its large extracellular domain contains eight fibronectin type III-like domains. Unlike many other Receptor PTPs, Sap-1 has a single, catalytically active tyrosine phosphatase domain, and is related to GLEPP-1, PTP-β, DEP-1 and PTPS31 (Hooft van Huijsduijnen 1998; Andersen et al. 2001). These “receptor-like” phosphatases share a common organization, which is characterized by an extracellular domain that includes Fibronectin-III-like repeats, and a single intracellular PTP domain. The fibronectin-like sequences are likely to be involved in cell-cell interactions.

No ligand for Sap-1 has been identified so far.

Seo et al. (1997) analyzed Sap-1 expression. No Sap-1 mRNA could be detected in healthy pancreas or colon, but mRNA and protein were highly expressed in pancreatic and colorectal cancer cells. Sap-1 expression was examined by immunohistochemistry in biopsies and its overexpression was found to correlate with the progression from adenomas with mild dysplasia into adenocarcinomas (Seo et al. 1997). Overexpression studies suggest p130^{cas} as a substrate for Sap-1 (Noguchi et al. 2001).

A number of PTPs can be regulated by their extracellular domains. Some type I “Receptor-” PTPs are known to dimerize (den Hertog et al. 1999; Meng et al. 2000). For PTP-α and CD45, it has been shown that this dimerization results in the repression of their intracellular catalytic domains (Majeti and Weiss, 2001). The only Receptor-PTP for which a ligand has been discovered, PTP-ξ, also belongs to this latter category (Meng et al. 2000). Binding of pleiotrophin to PTP-ξ results in receptor dimerization, inactivation of the enzyme and increased β-catenin phosphorylation and cell proliferation. All three examples involve PTPs with two catalytic-like domains, of which only the N-terminal domain (which is proximal to the plasma membrane) has activity.

However, for the group of phosphatases including Sap-1, PTP-β, DEP-1, GLEPP1 and PTPS31, the role of the extracellular domain has not been defined. No ligands were identified so far. It is unknown whether they are capable of dimerization, and what impact any such dimerization would have on phosphatase activity.

### SUMMARY OF THE INVENTION

The present invention is based on the finding that Sap-1 forms dimers resulting in a decrease of intracellular catalytic activity. Therefore, the invention relates to the use of a cross-linking agent for protein tyrosine phosphatase Sap-1 for treatment and/or prevention of cancer. The cross-linker is preferably used for treatment or prevention of src-associated and gastrointestinal cancers.

### BRIEF DESCRIPTION OF THE DRAWINGS

FIG. 1: (A) Sap-1 aminoacid sequence (corrected). Single underlined: signal peptide; double underlined: Transmembrane sequence. Bold: Catalytic domain. In some cDNAs I₂₄₄ is a valine instead (polymorphism). (B) PTP-Sap1 constructs used for the present invention. The GST- construct is expressed in bacteria. The other constructs are expressed in mammalian cells. The bold horizontal line represents the plasma membrane. FL: full length, HA: hemagglutinin tag, FLEX: full length extracellular domain, Myc-His tag: c-Myc tag plus histidine₆ tag, FN III: fibronectin type III repeats and fatty acid: myristilation site of Lck fused to the cytoplasmic domain of PTP-Sap1.

FIG. 2: Analysis of PTP-Sap1 expression. (A) Northern blot, probed with human Sap-1 (top) or β-actin (bottom, control) probes. On the blot was a total of 20 μg total RNA from human cell lines: HUVEC (1), Large cell lung carcinoma, ATCC HTB-177 (2), Lung adenocarcinoma, CRL-5800 (3), Ovary adenocarcinoma, HTB-161 (4), Colon adenocarcinoma CCL-224 (5), Colon adenocarcinoma CCL-218 (6), and Embryonal rhabdomyosarcoma, CCL-136 (7). (B) Multiple tissue array with poly(A)RNA from human tissues. Sap-1 hybridization as for (A) (C) TaqMan analysis for various tissues. WiDr is a colon adenocarcinoma cell line. Expression is given as a percentage of the signal for GAPDH mRNA.

FIG. 3: Peptide dephosphorylation assay. Phosphopeptides including the C-terminal autoinhibitory phosphotyrosine of src and lck were dephosphorylated with human PTPs.

FIG. 4: Src is recognized and activated by Sap1. (A) Pull down assay of c-Src by a substrate-trapping Sap1 mutant. WCL: whole cell lysate, FT: flow through and PD: pulled down. (B) Dephosphorylation of src by Sap-1. Src was immunoprecipitated from overexpressing cells and incubated with 1 mg GST or GST-Sap1. Phosphatase-treated src is no longer tyrosine phosphorylated. (C) Activation of c-src by Sap1. Immoprecipated src from overexpressing cells was incubated with GST-Sap1 (WT), GST-PTP-β or GST. Kinase activity was subsequently measured by adding MBP (myelin basic protein) kinase substrate. Right panel, in a separate experiment the GST-Sap1 DA trapping mutant was used. (D) In vivo activation of src. 3T3 cells were co-transfected with expression vectors for src plus empty vector or vectors expressing HA-tagged Sap-1 without the extracellular domain “intra”: FIG. 1 B) or as full-length (FL). Src was immunoprecipitated and tested for kinase activity (left panel) or controlled for expression of src (middle panel) and Sap1 (right panel, WCL whole cell lysate). (E) In vivo interaction between c-src and Sap-1, and phosphorylation of Sap-1. Cells were transfected with vectors expressing the wt (wild type) or trapping mutants of Sap-1, plus vectors expressing wt or Tyr₅₃₀˜mutated src (src Y530F). The HA (hemagglutinin)-tagged Sap protein was immunoprecipitated and the (co-)immunoprecipitate was analyzed by Western blotting and three antibodies. IB=immunoblot; IP=immunoprecipitation.

FIG. 5: Full-length Sap-1 can be chemically cross-linked. (A) COS7 cells were transfected with expression vectors for HA-tagged, full-length (FL) or ECD-deleted Sap1 (“intra”), and treated with BS3 cross-linking agent. (B) Cross-linking with DSS, a chemical cross-linker that is cell-permeable. Sap-1 m.a. is a membrane-associated version of Sap-1. Cross-linked products are indicated by stars.

FIG. 6: Involvement of cysteine(s) in Sap-1 multimerization. (A) COS7 cells transfected with Sap-1 constructs or vector were lysed in RIPA buffer containing IODA to block free SH-groups. The protein extracts were electrophoresed under reducing or non-reducing conditions. A BS3 cross-linking experiment was performed in parallel. (B) Left panel: COS7 overexpressing PTP-Sap1 were incubated for 30 min. with the indicated concentration of Dithiothreitol (DTT) and lysed for analysis as described in (A). Right panel: After incubation for 30 min., DTT was washed out and cells were incubated for a further 15 min. in DTT-free medium.

FIG. 7: Sap-1 forms dimers. Left panel: Cells were transfected to express full-length, His6-tagged Sap-1 (Sap-1 FLEX) plus various HA-tagged Sap-1 constructs. HA-tagged protein was immunoprecipitated, and the presence of the His-tagged Sap-1 detected with an antibody. Right panel: Control. The same samples as shown in the left panel were tested on Western blot for the expression of HA-tagged constructs.

FIG. 8: Redundancy in ECD-mediated dimerization. (A) FLEX-C-term construct was expressed in COS 7 treated or not with BS3 cross-linker. The construct is detected on Western blot with an anti-Myc antiserum. (B) PTP-Sap1 FL-HA was co-transfected with FLEX-C-terminal and FLEX-N-terminal and control constructs. Following immunoprecipitation with anti-Myc antibody, protein was visualised with co-imunoprecipitated anti-His (FLEX constructs) or with anti-HA (control for Sap1 FLHA).

FIG. 9: Sap-1 ECD-induced dimerization reduces enzymatic activity (A) Membrane fractions (1 mg) from cells transfected with various Sap-1 constructs were tested for P-SRCY530 dephosphorylation (B) Sap1intraHA containing membrane fractions were tested for phosphatase activity in presence of varying amounts of anti-HA-12CA5 monoclonal antibody.

### DETAILED DESCRIPTION OF THE INVENTION

The present invention is based on the finding that Sap-1 mRNA is expressed at very low levels in normal tissues and primary cells, but highly overexpressed in various intestinal carcinoma lines. It has further been shown that among a large panel of other phosphatases, the Sap-1 catalytic domain is unique in its capacity to dephosphorylate phosphopeptides corresponding to the C-terminal c-src and c-lck autoinhibitory control sequences. In addition this, the inventors of the present invention have demonstrated that Sap-1 is able to dephosphorylate and activate c-src in cells.

In accordance with the present invention, it has been surprisingly found that Sap-1 is able to form homodimers, that the fibronectin III-like extracellular domain is involved in dimerization, and that dimerization can be disrupted by reduction (opening) of cysteine-bridges. It was further shown that dimerization results in a decrease of intracellular catalytic activity of Sap-1.

Therefore, the present invention relates to the use of a cross-linking agent for protein tyrosine phosphatase Sap-1 for the preparation of a medicament for treatment and/or prevention of cancer. The cross-linker may cross-link two or more Sap-1 molecules present on cancer cells, thus reducing Sap-1 activity and intracellular effects resulting from Sap-1 activity.

The term “treatment” within the context of this invention refers to any beneficial effect on progression of disease, including attenuation, reduction, decrease or diminishing of the pathological development after onset of disease.

The term “prevention” within the context of this invention refers not only to a complete prevention of a certain effect, but also to any partial or substantial prevention, attenuation, reduction, decrease or diminishing of the effect before or at early onset of disease.

A “cross-linker” may be any chemical molecule, protein, polypeptide, fragment, mutein, derivative, or salt thereof, that cross-links two or more molecules of the protein tyrosine phosphatase Sap-1, thereby reducing, attenuating, decreasing or diminishing Sap-1 phosphatase activity.

In the context of the present invention Sap-1 cross-linking was shown to reduce its catalytic activiy, thereby opening new strategies for treating or preventing cancers wherein reduction of Sap-1 phosphatase activity has therapeutic value.

Sap-1 is part of a PTP sub-family whose members have a similar overall structural organization. Other members of this protein families are: GLEPP-1, PTP-β, DEP-1 and PTPS31. Cross-linkers of these receptor-like phosphatases may also be used for the treatment and/or prevention of cancers in which the activity of GLEPP-1, PTP-β, DEP-1 and PTPS31 is required to be reduced, respectively.

It has been found in the context of the present invention that Sap-1 is a phosphatase catalysing dephosphorylation, thereby leading to activation, of protein src and other src-like kinases. It is well known that src over-activity is involved in the development of cancer.

Therefore, in a preferred embodiment of the invention, the cancer is a cancer that is associated with src kinase activity. Src activity has been associated with a number of cancers, such as colon cancer, rectal cancer, prostate cancer and breast cancer, in particular in metastasizing cancers. The cross-linkers of the invention are particularly useful for treatment or prevention of these cancers.

Sap-1 has been found to be overexpressed in gastrointestinal cancers, in particular in pancreatic and colorectal cancer cells (Matozaki et al., 1994, Seo et al., 1997).

Therefore, in a further preferred embodiment of the invention, the cancer is a gastrointestinal cancer.

Gastrointestinal cancer or tumor may be any cancer or tumor originating, or being present in the gastrointestinal tract.

Preferably, the gastrointestinal cancer is selected from the group consisting of esophageal tumors, stomach cancer, small-bowel tumors, large-bowel tumors, and pancreatic cancers.

Esophageal cancers have been described above in the background of the invention. They include, but are not limited to epidermoid carcinoma, adenocarcinoma, lymphoma, leiomyosarcoma, and metastatic cancer.

Stomach cancers may be gastric adenocarcinoma, lymphomas or leiomyosarcomas.

Small-bowel cancer or small-bowel tumors have been described above in the “Backgound of the invention”. They include benign and maglignant tumors, comprising jejunal and ileal tumors, leiomyomas, lipomas, neurofibromas, and fibromas, polyps, vascular tumors, hereditary hemorrhagic telangiectasia (Rendu-Osler-Weber syndrome), hemangiomas, Malignant tumors of the small bowel include e.g. adenocarcinoma, which may occur in the presence of Crohn's disease, primary malignant lymphoma, carcinoid tumors, or multiple tumors, Kaposi's sarcoma, lymphocytic leukemia, non-Hodgkin's lymphoma, Hodgkin's disease, or adenocarcinoma of the gastrointestinal tract.

Large-bowel tumors have been described in the “Background of the invention”. Large-bowel cancer may be polyps, colorectal or anorectal cancer, adenocarcinoma of the colon and rectum, squamous cloacogenic carcinoma, melanoma, lymphoma, and various sarcomas. They further include basal cell carcinoma, Bowen's disease (intradermal carcinoma), extramammary Paget's disease, cloacogenic carcinoma, and malignant melanoma.

Pancreatic cancer include exocrine tumors and endocrine tumors, including but not limited to the tumors that have been described in detail above in the “background of the invention”.

In a preferred embodiment, the cross-linking agent is a proteinaceous cross-linker. “Proteinaceous cross-linkers”, as used herein, refers to proteins, polypeptides and peptides useful for cross-linking two or more Sap-1 molecules, preferably via their extracellular domains, in order to reduce Sap-1 catalytic activity.

In a further preferred embodiment, the cross-linking agent is an antibody, preferably a monoclonal antibody.

In yet further preferred embodiments, the cross-linking agent is a humanized antibody, preferably a human antibody or an antibody produced by animals that have been genetically reconstituted with a human immunesystem.

As shown in the examples of the present application, the Fibronectin-type III like repeats of the Sap-1 extracellular domain are believed to participate in homophilic interaction between Sap-1 molecules. Therefore, the cross-linking antibody of the present invention is preferably binding to a Fibronectin-like domain of Sap-1.

The present invention further relates to the use of an inhibitor or Sap-1 for prevention or treatment of cancer, in particular of the cancer described in detail in the “Background of the invention”. The inhibitor may be any kind of inhibitor such as e.g. a small molecular weight compound, an antisense RNA or interfering RNA (RNAi) Preferably, the inhibitor is an antibody binding to Sap-1. The antibody may preferably bind to the extracellular portion of Sap-1, such as e.g. to one or more of the Fibronectin-type III like repeats of Sap-1. Binding of the Sap-1 antibody of the invention leads to inhibition of the biologic activity of Sap-1 PTP, e.g. of catalysing dephosphorylation of src-kinase. Such an antibody may be monovalent, bivalent or multivalent, it may have cross-linking activity or not, as long as it blocks the catalytic activity of Sap-1. The antibody may e.g. bind to a single Sap-1 extracellular chain thereby inducing a conformational change leading to inhibition or inactivation of Sap-1 catalytic activity.

Antibodies used in accordance with the present invention can be polyclonal, such as antibodies produced in the rabbit, or monoclonal antibodies. They may recognize full length human Sap-1, or a fragment thereof, preferably a fragment comprising the extracellular domain of human Sap-1, such as a soluble fragment comprising one or more of the eight Fibronectin-type III like repeats present in the full-length extracellular Sap-1 domain, for example.

Preferably, the invention relates to the use of an antibody selected from the group consisting of polyclonal, monoclonal, chimeric, humanized, human or anti-anti-idiotype antibodies or fragments thereof which recognize and bind to the human Sap-1 or a fragment of the human Sap-1.

The term “monoclonal antibody” (mAb) is meant to include monoclonal antibodies, chimeric, humanized antibodies, human antibodies, antibodies to anti-idiotypic antibodies (anti-anti-ld antibody) that can be labeled in soluble or bound form, as well as fragments thereof provided by any known technique, such as, but not limited to enzymatic cleavage, peptide synthesis or recombinant techniques.

A monoclonal antibody contains a substantially homogeneous population of antibodies specific to antigens, which population contains substantially similar epitope binding sites. mAbs may be obtained by methods known to those skilled in the art. See, for example Kohler and Milstein, Nature, 256:495-497 (1975); U.S. Pat. No. 4,376,110; Ausubel et al., eds., Harlow and Lane ANTIBODIES: A LABORATORY MANUAL, Cold Spring Harbor Laboratory (1988); and Colligan et al., eds., Current Protocols in Immunology, Greene Publishing Assoc. and Wiley Interscience N.Y., (1992-1996). Such antibodies may be of any immunoglobulin class including IgG, IgM, IgE, IgA, and any subclass thereof. A hybridoma producing a mAb of the present invention may be cultivated in vitro, in situ or in vivo. Production of high titers of mAbs in vivo or in situ makes this the presently preferred method of production.

Chimeric antibodies are molecules of which different portions are derived from different animal species, such as those having the variable region derived from a murine mAb and a human immunoglobulin constant region. Chimeric antibodies are primarily used to reduce immunogenicity in application and to increase yields in production, for example, where murine mAbs have higher yields from hybridomas but higher immunogenicity in humans, such that human/murine chimeric mAbs are used. Chimeric antibodies and methods for their production are known in the art (Cabilly et al., European Patent Application 125023 (published Nov. 14, 1984); Taniguchi et al., European Patent Application 171496 (published Feb. 19, 1985); Morrison et al., European Patent Application 173494 (published Mar. 5, 1986); Neuberger et al., PCT Application WO 8601533, (published Mar. 13, 1986); Kudo et al., European Patent Application 184187 (published Jun. 11, 1986); Robinson et al., International Patent Application No. WO8702671 (published May 7, 1987); Riechmann et al. and Harlow and Lane, ANTIBODIES: A LABORATORY MANUAL, supra.

“Human antibodies” are molecules containing both the variable and constant region of the human immunoglobulin. Fully human antibodies are particularly suitable for therapeutic use, since anti-idiotypic immunogenicity should be significantly reduced or ideally be absent. One method for the preparation of fully human antibodies consist of “humanization” of the mouse humoral immune system, i.e. production of mouse strains able to produce human Ig (Xenomice), by the introduction of human immunoglobulin (Ig) loci into mice in which the endogenous Ig genes have been inactivated. The Ig loci are exceedingly complex in terms of both their physical structure and the gene rearrangement and expression processes required to ultimately produce a broad immune response. Antibody diversity is primarily generated by combinatorial rearrangement between different V, D, and J genes present in the Ig loci. These loci also contain the interspersed regulatory elements, which control antibody expression, allelic exclusion, class switching and affinity maturation. Introduction of unrearranged human Ig transgenes into mice has demonstrated that the mouse recombination machinery is compatible with human genes. Furthermore, hybridomas secreting antigen specific hu-mAbs of various isotypes can be obtained by Xenomice immunisation with antigen.

Fully human antibodies and methods for their production are known in the art (Mendez et al (1997); Buggemann et al (1991); Tomizuka et al., (2000) Patent WO 98/24893).

An anti-idiotypic (anti-Id) antibody is an antibody, which recognizes unique determinants generally associated with the antigen-binding site of an antibody. An Id antibody can be prepared by immunizing an animal of the same species and genetic type (e.g. mouse strain) as the source of the mAb to which an anti-Id is being prepared. The immunized animal will recognize and respond to the idiotypic determinants of the immunizing antibody by producing an antibody to these idiotypic determinants (the anti-Id antibody). See, for example, U.S. Pat. No. 4,699,880.

The anti-Id antibody may also be used as an “immunogen” to induce an immune response in yet another animal, producing a so-called anti-anti-Id antibody. The anti-anti-Id may be epitopically identical to the original mAb, which induced the anti-Id. Thus, by using antibodies to the idiotypic determinants of a MAb, it is possible to identify other clones expressing antibodies of identical specificity.

Accordingly, mAbs generated against SAP-1, its isoforms, analogs, fragments or derivatives thereof, may be used to induce anti-Id antibodies in suitable animals, such as BALB/c mice. Spleen cells from such immunized mice are used to produce anti-Id hybridomas secreting anti-Id mabs. Further, the anti-Id mAbs can be coupled to a carrier such as keyhole limpet hemocyanin (KLH) and used to immunize additional BALB/c mice. Sera from these mice will contain anti-anti-ld antibodies that have the binding properties of the original mAb specific for an epitope of the above SAP-1 protein, or analogs, fragments and derivatives thereof.

The anti-Id mAbs thus have their own idiotypic epitopes, or “idiotopes” structurally similar to the epitope being evaluated.

The term “monoclonal antibody” is also meant to include both intact molecules as well as fragments thereof, such as, for example, F(ab) or F(ab′)2, which are capable of binding antigen. Such fragments are typically produced by proteolytic cleavage, using enzymes such as e.g. pepsin or papain. F(ab) or F(ab′)2 fragments, clear more rapidly from the circulation, and may have less non-specific tissue binding than an intact antibody (Wahl et al. 1983).

The use of cross-linking antibodies is particularly preferred in accordance with the present invention. Because natural immunoglobulins have two antigen-recognizing domains, they have an intrinsic property to dimerize (multimerize) antigens. Suitable cross-linking antibodies may be tested in a suitable cellular readout system, in order to assess the antibody's capability of inhibiting Sap-1 enzymatic activity. Alternatively, strongly binding single-chain immunoglobulins, or Mabs that bind Sap-1 well but fail to induce the right type of di- or multimerization can be cross-linked using a variety of chemical procedures. Many techniques exist that covalently link Mabs to enzymes so that they can be used in immunofluorescence or ELISA. These procedures can be used to created multimeric Mabs as well.

In an alternative embodiment of the invention, the cross-linking agent is a soluble protein comprising all, or a portion of, the extracellular part of Sap-1. The soluble protein may preferably comprise one, two, three, four, five, six, seven or eight Fibronectin-type III like domains of Sap-1.

The soluble protein is preferably produced recombinantly. It may e.g. be produced in a eukaryotic expression system, preferably in a mammalian cell, such as in CHO cells.

The person skilled in the art will appreciate that there may be many further proteinaceous cross-linkers that can be used in frame of the present invention. Such further cross-linkers may e.g. be proteins that bind to and cross-link Sap-1 molecules, such as naturally occurring, preferable human proteins binding to Fibronectin, like tenascin, collagen, or fragments thereof, or soluble fragments of integrin receptors.

A mutein, fused protein, functional derivative, active fraction or salt of a cross-linking antibody, or a soluble Sap-1-derived protein, capable of cross-linking the extracellular domain of Sap-1, are also preferred according to the present invention.

As used herein the term “muteins” refers to analogs of a proteinaceous cross-linker, in which one or more of the amino acid residues of the proteinaceous cross-linker are replaced by different amino acid residues, or are deleted, or one or more amino acid residues are added. These muteins are prepared by known synthesis and/or by site-directed mutagenesis techniques, or any other known technique suitable therefor.

Muteins in accordance with the present invention include proteins encoded by a nucleic acid, such as DNA or RNA, which hybridizes to DNA or RNA, which encodes a proteinaceous cross-linker, under stringent conditions. The term “stringent conditions” refers to hybridization and subsequent washing conditions, which those of ordinary skill in the art conventionally refer to as “stringent”. See Ausubel et al., Current Protocols in Molecular Biology, supra, Interscience, N.Y., §§6.3 and 6.4 (1987, 1992), and Sambrook et al., supra. Without limitation, examples of stringent conditions include washing conditions 12-20° C. below the calculated Tm of the hybrid under study in, e.g., 2×SSC and 0.5% SDS for 5 minutes, 2×SSC and 0.1% SDS for 15 minutes; 0.1×SSC and 0.5% SDS at 37° C. for 30-60 minutes and then, a 0.1×SSC and 0.5% SDS at 68° C. for 30-60 minutes. Those of ordinary skill in this art understand that stringency conditions also depend on the length of the DNA sequences, oligonucleotide probes (such as 1040 bases) or mixed oligonucleotide probes. If mixed probes are used, it is preferable to use tetramethyl ammonium chloride (TMAC) instead of SSC. See Ausubel, supra.

Any such mutein preferably has a sequence of amino acids sufficiently duplicative of that of the original proteinaceous cross-linker, such as to be capable of cross-linking Sap-1 and thereby reducing or diminishing Sap-1 activity. The mutein can be defined by its substantial binding activity to Sap-1, in particular the extracellular domain of Sap-1, and its capacity to cross-link Sap-1 molecules and thereby reducing Sap-1 phosphatase activity, such as src dephosphorylation, for instance.

In a preferred embodiment, any such mutein has at least 40% identity or homology with the sequence of a proteinaceaous cross-linker, such as a recombinant antibody directed against the extracellular domain of Sap-1, or a soluble portion of the extracellular domain of Sap-1. More preferably, it has at least 50%, at least 60%, at least 70%, at least 80% or, most preferably, at least 90% identity or homology thereto.

Muteins of proteinaceous cross-linkers of Sap-1, which can be used in accordance with the present invention, or nucleic acid coding therefor, include a finite set of substantially corresponding sequences as substitution peptides or polynucleotides which can be routinely obtained by one of ordinary skill in the art, without undue experimentation, based on the teachings and guidance presented herein.

Preferred changes for muteins in accordance with the present invention are what are known as “conservative” substitutions. Conservative amino acid substitutions may include synonymous amino acids within a group which have sufficiently similar physicochemical properties that substitution between members of the group will preserve the biological function of the molecule (Grantham, 1974). It is clear that insertions and deletions of amino acids may also be made in the above-defined sequences without altering their function, particularly if the insertions or deletions only involve a few amino acids, e.g., under thirty, and preferably under ten, and do not remove or displace amino acids which are critical to a functional conformation, e.g., cysteine residues. Proteins and muteins produced by such deletions and/or insertions come within the purview of the present invention.

Preferably, the synonymous amino acid groups are those defined in Table 1. More preferably, the synonymous amino acid groups are those defined in Table 2; and most preferably the synonymous amino acid groups are those defined in Table 3.

TABLE 1
Preferred Groups of Synonymous Amino Acids

| Amino Acid | Synonymous Group |
|---|---|
| Ser | Ser, Thr, Gly, Asn |
| Arg | Arg, Gln, Lys, Glu, His |
| Leu | Ile, Phe, Tyr, Met, Val, Leu |
| Pro | Gly, Ala, Thr, Pro |
| Thr | Pro, Ser, Ala, Gly, His, Gln, Thr |
| Ala | Gly, Thr, Pro, Ala |
| Val | Met, Tyr, Phe, Ile, Leu, Val |
| Gly | Ala, Thr, Pro, Ser, Gly |
| Ile | Met, Tyr, Phe, Val, Leu, Ile |
| Phe | Trp, Met, Tyr, Ile, Val, Leu, Phe |
| Tyr | Trp, Met, Phe, Ile, Val, Leu, Tyr |
| Cys | Ser, Thr, Cys |
| His | Glu, Lys, Gln, Thr, Arg, His |
| Gln | Glu, Lys, Asn, His, Thr, Arg, Gln |
| Asn | Gln, Asp, Ser, Asn |
| Lys | Glu, Gln, His, Arg, Lys |
| Asp | Glu, Asn, Asp |
| Glu | Asp, Lys, Asn, Gln, His, Arg, Glu |
| Met | Phe, Ile, Val, Leu, Met |
| Trp | Trp |

TABLE 2
More Preferred Groups of Synonymous Amino Acids

| Amino Acid | Synonymous Group |
|---|---|
| Ser | Ser |
| Arg | His, Lys, Arg |
| Leu | Leu, Ile, Phe, Met |
| Pro | Ala, Pro |
| Thr | Thr |
| Ala | Pro, Ala |
| Val | Val, Met, Ile |
| Gly | Gly |
| Ile | Ile, Met, Phe, Val, Leu |
| Phe | Met, Tyr, Ile, Leu, Phe |
| Tyr | Phe, Tyr |
| Cys | Cys, Ser |
| His | His, Gln, Arg |
| Gln | Glu, Gln, His |
| Asn | Asp, Asn |
| Lys | Lys, Arg |
| Asp | Asp, Asn |
| Glu | Glu, Gln |
| Met | Met, Phe, Ile, Val, Leu |
| Trp | Trp |

TABLE 3
Most Preferred Groups of Synonymous Amino Acids

| Amino Acid | Synonymous Group |
|---|---|
| Ser | Ser |
| Arg | Arg |
| Leu | Leu, Ile, Met |
| Pro | Pro |
| Thr | Thr |
| Ala | Ala |
| Val | Val |
| Gly | Gly |
| Ile | Ile, Met, Leu |
| Phe | Phe |
| Tyr | Tyr |
| Cys | Cys, Ser |
| His | His |
| Gln | Gln |
| Asn | Asn |
| Lys | Lys |
| Asp | Asp |
| Glu | Glu |
| Met | Met, Ile, Leu |
| Trp | Met |

Examples of production of amino acid substitutions in proteins which can be used for obtaining muteins of proteinaceous cross-linkers, for use in the present invention include any known method steps, such as presented in U.S. Pat. Nos. 4,959,314, 4,588,585 and 4,737,462, to Mark et al; U.S. Pat. No. 5,116,943 to Koths et al., U.S. Pat. No. 4,965,195 to Namen et al; U.S. Pat. No. 4,879,111 to Chong et al; and U.S. Pat. No. 5,017,691 to Lee et al; and lysine substituted proteins presented in U.S. Pat. No. 4,904,584 (Shaw et al).

The term “fused protein” refers to a polypeptide comprising a proteinaceous cross-linker, or a mutein or fragment thereof, fused with another protein, which, e.g., has an extended residence time in body fluids. A proteinaceous cross-linker of Sap-1 may thus be fused to another protein, polypeptide or the like, e.g., an immunoglobulin or a fragment thereof. Methods for making immunoglobulin fusion proteins are well known in the art, such as the ones described in WO 01/03737, for example. The person skilled in the art will understand that the resulting fusion protein of the invention retains the cross-linking activity for Sap-1, and in particular its attenuation of Sap-1 activity. The fusion may be direct, or via a short linker peptide which can be as short as 1 to 3 amino acid residues in length or longer, for example, 13 to 20 amino acid residues in length. Said linker may be a tripeptide of the sequence E-F-M (Glu-Phe-Met), for example, or a β-amino acid linker sequence comprising Glu-Phe-Gly-Ala-Gly-Leu-Val-Leu-Gly-Gly-Gln-Phe-Met introduced between the peptide sequence of a proteinaceous cross-linker and the immunoglobulin sequence. The resulting fusion protein may have improved properties, such as an extended residence time in body fluids (half-life), increased specific activity, increased expression level, or the purification of the fusion protein is facilitated.

In a preferred embodiment, the proteinaceous cross-linker is fused to the constant region of an Ig molecule. Preferably, it is fused to heavy chain regions, like the CH2 and CH3 domains of human IgG1 or IgG2, for example. Other isoforms of Ig molecules are also suitable for the generation of fusion proteins according to the present invention, such as isoforms IgG₂ or IgG₄, or other Ig classes, like IgM or IgA, for example.

“Functional derivatives” as used herein cover derivatives of proteinaceous cross-linkers and their muteins and fused proteins, which may be prepared from the functional groups which occur as side chains on the residues or the N- or C-terminal groups, by means known in the art, and are included in the invention as long as they remain pharmaceutically acceptable, i.e. they do not destroy the activity of the protein which is cross-linking Sap-1 and thereby reducing Sap-1 dephosphorylation activity.

These derivatives may, for example, include polyethylene glycol side-chains, which may mask antigenic sites and extend the residence of a proteinaceous cross-linker in body fluids. Other derivatives include aliphatic esters of the carboxyl groups, amides of the carboxyl groups by reaction with ammonia or with primary or secondary amines, N-acyl derivatives of free amino groups of the amino acid residues formed with acyl moieties (e.g. alkanoyl or carbocyclic aroyl groups) or O-acyl derivatives of free hydroxyl groups (for example that of seryl or threonyl residues) formed with acyl moieties.

As “active fractions” of a proteinaceous cross-linker, a mutein or fused protein thereof, the present invention covers any fragment or precursors of the polypeptide chain of the protein molecule alone or together with associated molecules or residues linked thereto, e.g., sugar or phosphate residues, or aggregates of the protein molecule or the sugar residues by themselves, provided said fraction has substantial cross-linking activity vis-à-vis Sap-1.

Preferably, the cross-linking agent is directed against a Fibronectin-type III like domain.

It is further preferred to use a cross-linking agent that is a bivalent or multivalent.

In yet a further preferred embodiment, the cross-linking agent is a functional derivative comprising at least one moiety attached to one or more functional groups, which occur as one or more side chains on the amino acid residues.

Functional derivatives of Sap-1 cross-linkers may be conjugated to polymers in order to improve the properties of the cross-linker, such as the stability, half-life, bioavailability, tolerance by the human body, or immunogenicity. To achieve this goal, the cross-linker may be linked to Polyethlyenglycol (PE.G.). PEGylation may be carried out by known methods, such as the ones described in WO 92/13095, for example.

Therefore, in a preferred embodiment of the present invention, a functional derivative of a cross-linker of the present invention comprises a polyethylene moiety, i.e. it is PEGylated.

The invention further relates to the use of an expression vector comprising the coding sequence of a proteinaceous cross-linker in the preparation of a medicament for the prevention and/or treatment of a peripheral vascular disease. Thus, a gene therapy approach is considered in order to deliver the proteinaceous cross-linker to the site where it is required. In order to treat and/or prevent cancer. The gene therapy vector comprising the sequence of proteinaceous cross-linker may be injected directly into the diseased tissue, for example, thus avoiding problems involved in systemic administration of gene therapy vectors, like dilution of the vectors, reaching and targeting of the target cells or tissues, and of side effects.

The Sap-1 cross-linker to be used in accordance with the present invention may be preferably administered as a pharmaceutical composition.

The definition of “pharmaceutically acceptable” is meant to encompass any carrier, which does not interfere with effectiveness of the cross-linking activity of the active ingredient and that is not toxic to the host to which it is administered. For example, for parenteral administration, the active protein(s) may be formulated in a unit dosage form for injection in vehicles such as saline, dextrose solution, serum albumin and Ringer's solution.

The active ingredients of the pharmaceutical composition according to the invention can be administered to an individual in a variety of ways. The routes of administration include intradermal, transdermal (e.g. in slow release formulations), intramuscular, intraperitoneal, intravenous, subcutaneous, oral, intracranial, epidural, topical, and intranasal routes. Any other therapeutically efficacious route of administration can be used, for example absorption through epithelial or endothelial tissues or by gene therapy wherein a DNA molecule encoding the active agent is administered to the patient (e.g. via a vector), which causes the active agent to be expressed and secreted in vivo. In addition, the protein(s) according to the invention can be administered together with other components of biologically active agents such as pharmaceutically acceptable surfactants, excipients, carriers, diluents and vehicles.

For parenteral (e.g. intravenous, subcutaneous, intramuscular) administration, the active protein(s) can be formulated as a solution, suspension, emulsion or lyophilized powder in association with a pharmaceutically acceptable parenteral vehicle (e.g. water, saline, dextrose solution) and additives that maintain isotonicity (e.g. mannitol) or chemical stability (e.g. preservatives and buffers). The formulation is sterilized by commonly used techniques.

The bioavailability of the active protein(s) according to the invention can also be ameliorated by using conjugation procedures which increase the half-life of the molecule in the human body, for example linking the molecule to polyethylenglycol, as described in the PCT Patent Application WO 92/13095.

The therapeutically effective amounts of the active component of the pharmaceutical composition will be a function of many variables, any residual cytotoxic activity exhibited by the antagonists, the route of administration, and the clinical condition of the patient.

A “therapeutically effective amount” is such that when administered, the Sap-1 cross-linker results in inhibition of the biological activity of Sap-1. The dosage administered, as single or multiple doses, to an individual will vary depending upon a variety of factors, including Sap-1 cross-linker pharmacokinetic properties, the route of administration, patient conditions and characteristics (sex, age, body weight, health, size), extent of symptoms, concurrent treatments, frequency of treatment and the effect desired. Adjustment and manipulation of established dosage ranges are well within the ability of those skilled in the art, as well as in vitro and in vivo methods of determining the inhibition of Sap-1 in an individual.

According to the invention, the Sap-1 cross-linker is used in an amount of about 0.001 to 100 mg/kg or about 0.01 to 10 mg/kg or body weight, or about 0.1 to 5 mg/kg of body weight or about 1 to 3 mg/kg of body weight or about 2 mg/kg of body weight.

The route of administration which is preferred according to the invention is administration by subcutaneous route. Intramuscular administration is further preferred according to the invention. In order to administer the Sap-1 cross-linker directly to the place of its action, it is also preferred to administer it topically.

In further preferred embodiments, the Sap-1 cross-linker is administered daily or every other day.

The daily doses are usually given in divided doses or in sustained release form effective to obtain the desired results. Second or subsequent administrations can be performed at a dosage which is the same, less than or greater than the initial or previous dose administered to the individual. A second or subsequent administration can be administered during or prior to onset of the disease.

According to the invention, the Sap-1 cross-linker can be administered prophylactically or therapeutically to an individual prior to, simultaneously or sequentially with other therapeutic regimens or agents (e.g. multiple drug regimens), in a therapeutically effective amount, in particular with a chemotherapeutic agent and/or in combination with radiotherapy and/or surgery. Active agents that are administered simultaneously with other therapeutic agents can be administered in the same or different compositions. Treatments and medicaments for specific types of cancer that are stipulated in the frame of the present invention have been described in detail above in the “Background of the Invention”. The person skilled in the art will appreciate that Sap-1 cross-linkers may be co-administered or combined with any specific cancer for which treatments are already known.

The invention further relates to a method for the preparation of a pharmaceutical composition comprising admixing an effective amount of a Sap-1 cross-linker with a pharmaceutically acceptable carrier.

The invention further relates to a method of treatment of cancer, comprising administering a pharmaceutically effective amount of a Sap-1 cross-linker, to a patient in need thereof.

Having now fully described this invention, it will be appreciated by those skilled in the art that the same can be performed within a wide range of equivalent parameters, concentrations and conditions without departing from the spirit and scope of the invention and without undue experimentation.

While this invention has been described in connection with specific embodiments thereof, it will be understood that it is capable of further modifications. This application is intended to cover any variations, uses or adaptations of the invention following, in general, the principles of the invention and including such departures from the present disclosure as come within known or customary practice within the art to which the invention pertains and as may be applied to the essential features hereinbefore set forth as follows in the scope of the appended claims.

All references cited herein, including journal articles or abstracts, published or unpublished U.S. or foreign patent application, issued U.S. or foreign patents or any other references, are entirely incorporated by reference herein, including all data, tables, figures and text presented in the cited references. Additionally, the entire contents of the references cited within the references cited herein are also entirely incorporated by reference.

Reference to known method steps, conventional methods steps, known methods or conventional methods is not any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various application such specific embodiments, without undue experimentation, without departing from the general concept of the present invention. Therefore, such adaptations and modifications are intended to be within the meaning an range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

### EXAMPLES

Methods

Antibodies, Peptides and Chemical Reagents:

Anti-HA: for Western Blot a mouse monoclonal anti-HA.11 (BAbCO) (recognizing the influenza hemagglutinin (HA) epitope YPYDVPDYA) or a rabbit polyclonal anti-HA (Santa Cruz) was used. For immunoprecipiation, it was found that the more efficient antibody was the anti-HA.11 (BAbCO). For antibody-induced cross-linking, the mouse monoclonal anti-HA5CA.12 (Roche) was used. Anti-Myc: mouse monoclonal anti-Myc9E10 (Santa-Cruz). Anti-SRC: anti-SRC327 is a gift from Dr. J. Brugge, rabbit polyclonal anti-SRCPAN, rabbit polyclonal anti-SRCPY530 (Biosource) and rabbit polyclonal anti-SRC (Santa-Cruz). Anti-His: mouse monoclonal anti-His (Dianova)

Northern Blot, RT-PCR and Multiple Tissue Array

Total RNA was isolated using Trizol® reagent (Life Technologies) following manufacturer's recommendations. Northern blot hybridization was as described (Sambrook et al. 1989). Briefly, 20 μg of RNA were run on a formaldehyde agarose gel in MOPS buffer and the material was filter-transferred into a nitrocellulose. After UV-cross-linking, the gel was blocked in ExpressHyb® solution (Clontech) and the probe (corresponding to the 900 bp Sap-1 catalytic domain) was added. The probe was prepared using a HighPrime® kit (Roche).

The Multiple Tissue Array (MTE, Clontech) hybridization followed the protocol instructions, except that Klenow Polymerase (NEB) was used. Radiographies of the membranes were visualized using Kodak X-OMAT films.

Real-Time PCR by Taqman

Reverse-transcription-PCR (RT-PCR) was performed using 1 μg RNA and the Omniscript® RT kit (Qiagen) using the manufacturers' recommendations. The cDNA obtained was further tested by amplifying actin cDNA using specific primers (5′-CCA GCT CAC CAT GGA TGA TG-3′ and 5′-CCT TAA TGT CAC GCA CGA TTT C-3′) in order to test the quality and possible contamination with genomic DNA (to this end, RNA only is incubated in the PCR mix). cDNA was amplified by TaqMan® using the manufacturer's CyberGreen protocol and Sap-1-specific primers 5′-CAT GCT GAC CAA CTG CAT GG and 5′-GCG AGT CCA GAG GCC AGT AA.

PCR primers for human Sap1 and GAPDH (house-keeping control) were designed using the Primer Express software from Perkin-Elmer Biosystems based on the published sequences: Sap1, reverse GCG AGT CCA GAG GCC AGT AA; forward CAT GCT GAC CAA CTG CAT GG; GAPDH, reverse GAT GGG ATT TCC ATT GAT GAC A; forward CCA CCC ATG GCA AAT TCC; intron-GAPDH, reverse CCT AGT CCC AGG GCT TTG ATT; forward CTG TGC TCC CAC TCC TGA TTT C. The specificity and the optimal primer concentration were tested on diluted series of plasmids with cDNA inserts. Potential genomic DNA (of cDNA) contamination was excluded by performing PCR reactions with specific intron-GAPDH primers. The absence of non-specific amplification was controlled by analyzing the PCR products on 3.5% agarose gels. SYBR Green Real-Time PCR reactions contained 25 μl SYBR Green PCR master mix (PE Biosystems) with 0.5 U AmpErase Uracil N-Glycosylase (UNG) and 20 μl of primers (300 nM). Template was 5 μl of RT-products; 0.5 ng total RNA from human tissues (ClonTech) or polyA⁺ for ovary, using PE Multiscribe enzyme). WiDr (Human Human colon adenocarcinoma, HT-29) was obtained from the ATCC. PCR was performed at 50° C. for 2 min (AmpErase UNG contaminant DNA digestion; (Udaykumar et al. 1993)), 95° C. for 10 min (for AmpliTaq Gold activation) and then run for 40 cycles at 95° C. for 15 sec, 60° C. for 1 min on the ABI PRISM 7700 Detection System. The reverse-transcribed cDNA samples were thus amplified and their Ct (cycle threshold) values were determined. All Ct values were normalized to the housekeeping gene GAPDH. A single specific DNA band for human Sap1 and GAPDH was observed using gel electrophoresis analysis.

Phosphopeptide Dephosphorylation Assay

The peptides tested corresponded to src (TSTEPQZQPGENL; Z=phosphotyrosine) and lck (FFTATE.G.QZQPQP) C-terminal sequences; both were purchased from Neosystems. The GST-PTP enzymes were made and tested using Malachite Green as an indicator for free phosphate as described earlier (Wälchli et al. 2000). Per assay 1.25 ng of PTP was tested with 200 μM phosphopeptide in 40 μl that also contained 50 mM HEPES pH 7.2, 1 mM EDTA, 1 mM DTT and 0.05% (v/v/) NP-40. After incubation (30 min. at room T) 100 μl BioMol Green (BioMol) was added, and after a further 20 min the OD₆₅₀ was measured. The OD was converted to free nmoles phosphate using a standard curve.

Cell Culture, Transfection and Retroviral Infection

Cells were grown under 5% CO₂ in Dulbecco's modified Eagle's medium, DMEM, with 10% FCS (COS 7, 3T3 cells) or minimum essential medium, MEM with 10% FCS and 1% non-essential amino acids (WiDr). Both media were supplemented with 10⁵ U/L penicillin and streptomycin and L-Glutamine. Transfections were performed with Fugene-6 reagent (Roche) following the manufacturer's instructions.

Pull-Down Experiments

Total cell lysate of COS 7 cells overexpressing c-Src WT or Y530F mutant were incubated for 1 hour in the presence of the GST-PTP trapping mutant or GST. The complex was isolated by adding glutathion-sepharose beads which were extensively washed. Finally, sample buffer was added and the mix was run on a SDS-PAGE. Detection was performed after transfer on PVDF membrane using anti-SRC antibody.

Plasmid Constructs

The PTP-Sap1 FL construct (Matozaki et al. 1994) was subcloned into several vectors using XbaI and HindIII restriction enzymes into the pcDNA4a vector (Invitrogene). The pcDNA4-Sap1intra HA clone is a fusion of the signal peptide sequence of PTP-Sap1, followed by a HA-tag and the PTP sequence from the N-terminal part of the TM. It was prepared as follows: the full cytoplasmic domain of PTP-sap1 was amplified by PCR (polymerase chain reaction) using Herculase Polymerase (Stratagene) and an antisense primer from the vector and a sense primer containing the HA-tag fused to the sequence encoding the five extracellular amino acids on the N-terminal side of the TM (5′-TAC CCA TAC GAC GTC CCA GAC TAC GCT CAC ACC GAG AGT GCA GGG GT-3′). From the other end, the signal peptide was amplified with a reverse primer fused to the HA-tag in the C-terminal part of the sequence (5′-AGC GTA GTC TGG GAC GTC GTA TGG GTA GGG GGC AGG CGC CCT GGC CCC T-3′) and the forward primer was from the vector. The two PCR products were mixed and amplified again with external primers. Two XbaI sites (one generated and the other one from the original vector) surrounding the amplicon were used to insert the intracellular part of PTP-Sap1 fused to the signal peptide and the HA-tag. The full length construct fused in C-terminal with an HA-tag was prepared with an antisense primer containing the sequence of a XhoI site, an HA-tag and the end of the PTP-Sap1 sequence with the STOP codon (5′-TAC TCG AGT TAA GCG TAG TCT GGG ACG TCG TAT GGG TAG ACC TCC MC TTG TGG GCC T-3′). The full length Sap1-HA fusion construct was amplified using a plasmid specific forward primer in a long run PCR using Herculase in the presence of 5% DMSO and “hotstart” conditions. The HA-tag construct in N-terminal could never be obtained. FLEX construct was obtained by amplifying the ECD sequence with a primer complementary to the intracellular proximal TM coding sequence (about 30 bp down stream) in order to keep the targeting sequence (5′-ATG MT TCA GCG GCC CAT CTG GCT GCC TCT TTC TCA GGA AGA AAA TCA-3′) and adding an EcoRI site. When the amplicon is fused to pcDNA4b, it is in frame with the two tags (His and c-Myc) and a stopcodon. The C- and the N-terminal deletion in FLEX were performed by digestion of the pcDNA4-FLEX construct using ECD coding sequence cutting enzymes that maintain the correct reading frame. XcmI (NEB) and PstI were used to digest pcDNA4-FLEX; the larger fragments (containing the truncated FLEX) were purified and ligated again, for C- and N-terminal constructs, respectively. The GST-Sap1 construction was described previously (Walchli et al. 2000). PTP-Sap1 ma (membrane associated) consists of the fusion of the PTP-Sap1 cytoplasmic region with a HA-tag in its C-terminal part (amplification from the pcDNA4-PTP-Sap1 FLHA construct). A primer containing the Lck myristilation site (underlined) and a start codon surrounded by a Kozak sequence ATA AGC TTA CCA TGG GCT GTG GCT GCA GCT CAC ACC CGG AAG ATG ACT GGA AGA GGA GGA ATA AGA AGA AG, was used with a vector primer to amplify the cytoplasmic fragment of PTP-Sap1. The amplicon was cloned into pcDNA4 using HindIII restriction site of the primer for the 5′ end ligation.

Site-directed mutagenesis. The exchange of (a) codon(s) was done as follows. PCR was performed with Pfu Polymerase (Promega) following a special program with an extension time relative to the size of the plasmid used (0.5 kb/minute) for 14 cycles. Following the amplification of 100 ng of plasmid, DpnI is incubated in the mix for 1 hour at 37° C. Ultracompetent cells (XL2-blue, Stratagene) were transformed with 1/10 of the PCR mix and plated in selective medium. For PTP-Sap1 site-directed mutagenesis, the pair of primers was the following: C747S (5′-CTC TGT GGT CAG CCA CAC CGA GAG T-3′ and 5′-CTC GGT GTG GCT GAC CAC AGA GTG A-3′), D986A (trapping mutant, 3′-GCC TGG CCG GCT CAC GGC GTT CCC T-5′ and 3′-AAC GCC GTG AGC CGG CCA GG-5′), Y1094F (3′-CGA GM GGA AGT CCC GTT TGA GGA T-5′ and 3′-CAT CCT CAA ACG GGA CTT CCT TCT C-5′), Y1002F (3′-TGT CGA AAA CCT CAT CTT CGA GM C-5′ and 3′-CGG CCA CGT TCT CGA AGA TGA GGT T-5′)

All these constructs were controlled by sequencing.

c-Src Constructs:

c-Src cDNA was cloned in different vectors, but mostly used in pcDNA4. The c-Src Y530F mutant was made using the method previously described with the following primers (5′-AGT TCC AGC CCG GGG AGA ACC TC-3′ and 5′-GAG GTT CTC CCC GGG CTG GAA CT-3′).

Immunoprecipitation (IP)

Every IP was performed with the same buffer; optimum antibody titers were empirically determined. Cell lysates were prepared as follows: cells were incubated in RIPA lysis buffer (PBS, 1% IGEPAL, 0.5% Na-deoxycholate, 0.1% SDS supplemented with a complete proteases inhibitors cocktail tablet (Roche) and sodium orthovanadate 1 mM) or NP-40 buffer for anti-myc (50 mM Tris pH 7.5, 150 mM NaCl, 1 mM EDTA, 10 mM NaF, 25 mM β-glycerolphosphate, 1 mM NaPyrophosphate, 1% NP-40 and supplemented with a complete proteases inhibitors cocktail tablet (Roche)), both buffers were used ice-cold. Cells were mechanically broken by one cycle of freeze-thawing and passed through a G21 syringe. Lysates were clarified by centrifugation (14,000 rpm) for 10 minutes at 4° C. and precleared with nonspecific antibody (Santa Cruz) and 20 μl of precoated proteinA/G sepharose beads (Santa-Cruz). The mix was then spun down and the supernatant was recovered and incubated with the specific antibody, precoated for 2 hours on protA/G beads, O/N at 4° C. The beads were finally washed once with the lysis buffer and twice with PBS-0.1% TritonX-100 (the volume of washes corresponds to 2 volumes of lysate). The samples are then used for Western Blot analysis or a kinase assay.

Western Blot

Proteins were separated on SDS-polyacryamide gel (PAGE), NuPAGE-gel system from Novex (Invitrogene), following the manufacturer's protocol. Proteins were transferred to PVDF membranes using a semi-dry transfer apparatus (Pharmacia). All buffers (running and transfer) were from Novex, but it was found that the efficiency of the transfer buffer is increased when used 2× with 10% methanol final. After staining of the membrane with Amido-Black (Sigma), the blot was blocked for one hour in a solution of PBS-0.2% Tween® and 5% fat free powder milk. Antibodies were diluted in the blocking buffer and incubated at room temperature (RT) for one hour. After both antibodies' incubation, the blot was washed at least three times with PBS-0.2% Tween® for 15 minutes. Bands were revealed with the ECL detection system (Amersham).

Kinase Assay

After the last wash of the IP, the kinase was resuspended in phosphatase buffer (20 mM Tris pH 7.5, 150 mM NaCl, 1 mM DTT and 1 mM EDTA). 1/10 of this solution was run on a SDS-PAGE to perform a control Western Blot, the rest was used for kinase assay. For in vitro GST-Sap1 mediated activation, 10 or 100 ng of the purified PTP was added to the IP sample and incubated for 30 minutes at 30° C. If the IP kinase came from cells overexpressing the PTP (cellular activation), this step was omitted and the IP sample was directly incubated with MBP, 1 mM sodium orthovanadate and 35 μCi μ-γ-ATP. In addition, the buffer was made to a final concentration of 5 mM MgCl₂, 2.5 mM MnCl₂ and 2.5 mM DTT. The reaction was stopped after 30 minutes at 30° C., by the addition of protein-sample buffer and run on a SDS-PAGE. Bands were visualized after autoradiography using Kodak X-OMAT films.

BS3, DSS and DTT Treatments

COS 7 cells were transfected with the indicated construct and left for 24 hours at 37° C. Medium was changed and a solution of BS3 (Pierce) in PBS (2.5 mg/ml) or PBS alone was added, and left for 1 hour at 4° C. The cross-linking reaction was stopped by the addition of PBS that contained 0.15 M Tris pH 7.5 for 15 minutes at 4° C. Cells were finally lysed and the protein content was used for further analysis. The treatment with DSS (Pierce) was the same except that the product was dissolved in DMSO at a concentration of 25 mM (10×). It was then diluted in PBS and cells were incubated with 0.1×DMSO.

Cells treated with DTT were serum-starved 8 hours after transfection (with PTP-Sap1 constructs or vector), and then incubated for 30 minutes in serum-free medium with various amount of DTT (Sigma). Before lysis in a buffer containing 150 mM iodoacetamide (“IODA” Sigma), cells were washed with ice cold PBS containing 50 mM iodoacetamide in order to block the free DTT. Samples were run under reducing and non-reducing conditions, with or without β-mercaptoethanol in the sample buffer, respectively.

Cellular Membrane Preparation and Measurement of Phosphatase Activity

Transfection was in 10 cm plates (5×10⁵ cells) and cells were used after 24 hours. Cells were washed twice with ice-cold PBS and then harvested in 10 ml PBS. After centrifugation at 1,600 rpm (in 15 ml Falcon tubes), they were resuspended in 500 μl collecting buffer (50 mM Tris pH=7.6, 150 mM NaCl, plus protease inhibitors cocktail). Following sonication (3 times 10 sec), samples were ultracentrifuged at 10⁵ g for 30 minutes at 4° C. The cytoplasmic fraction (supernatant) was recovered and glycerol was added to 20%. The pellet was rinsed with collecting buffer once and resuspended in collecting buffer with 1% Triton-X100. The solution was kept 1 hour on ice and ultracentrifuged again at 10⁵ g for 30 minutes. The membrane fraction (supernatant) was isolated and glycerol was added to 20%. Protein expression was checked by Western blot. The protein concentration was determined by the method of Bradford (BioRad). 1 μg of membrane extract and 100 μM of substrate peptide were mixed in assay buffer (50 mM Hepes pH 7.4, 0.05% NP-40 and 1 mM DTT); the reaction was stopped by the addition of Malachite reagent (BioMol) at different time points. All experiments were performed in duplicate. The 96-well plate was read at OD₆₀₅.

### Example 1

### Corrected Sap-1 Sequence

Sap-1 Sequence and Expression

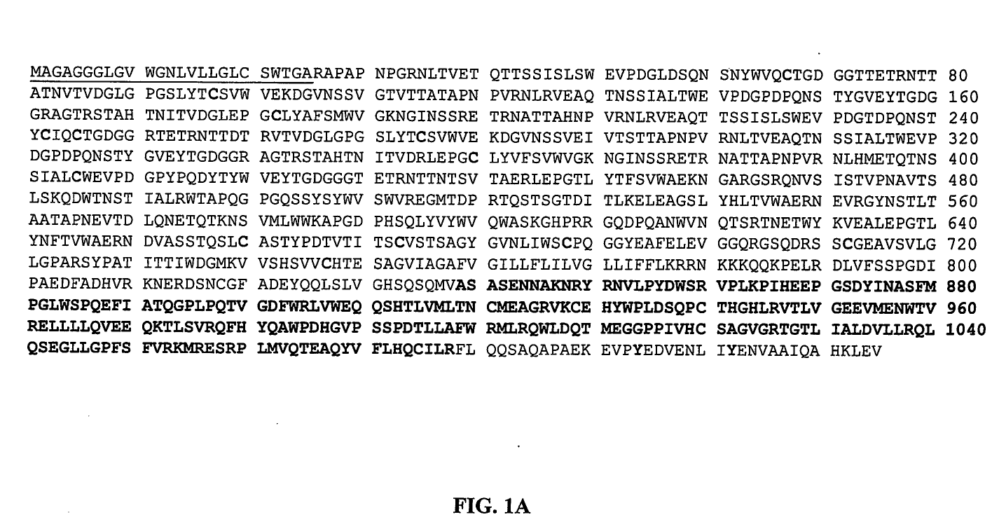

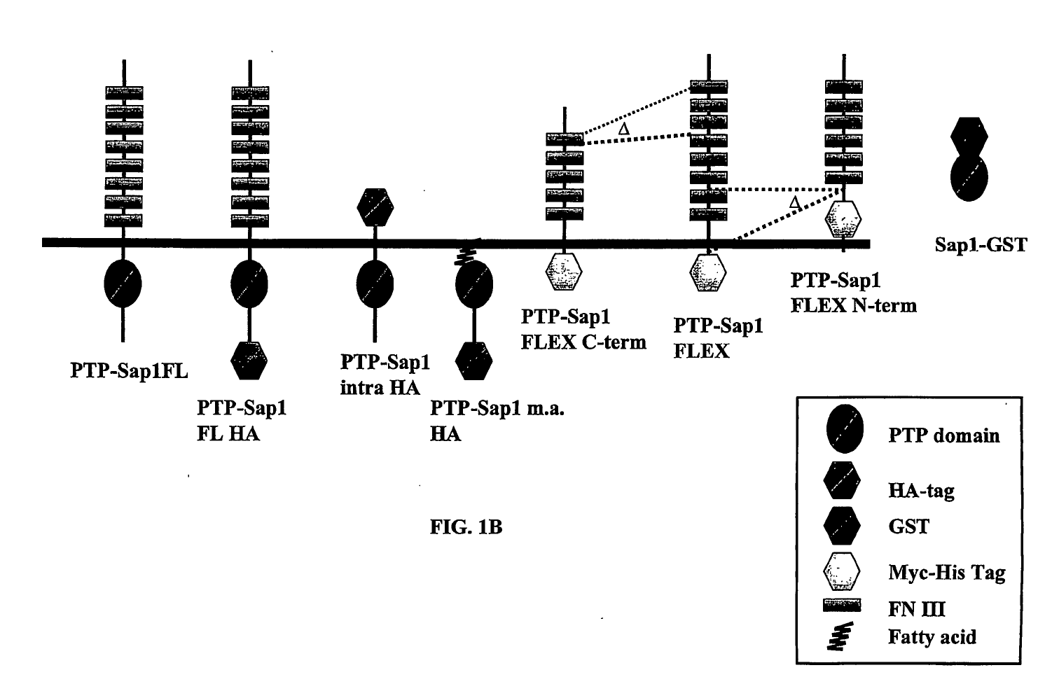

In the process of construction full-length Sap-1 expression vectors sequencing, errors were corrected in the previously published sequence. FIG. 1A shows the corrected, full-length Sap-1 amino acid sequence. FIG. 1B shows an overview over the different Sap-1 recombinant proteins that were used in the present examples.

### Example 2

### mRNA Expression of Sap-1 in Human Tissues

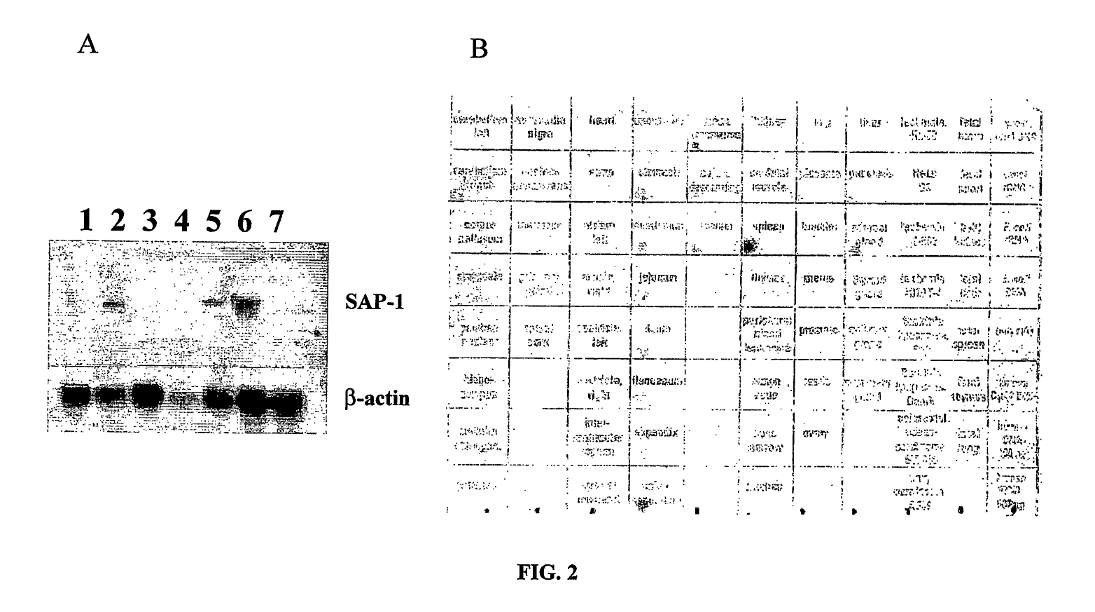

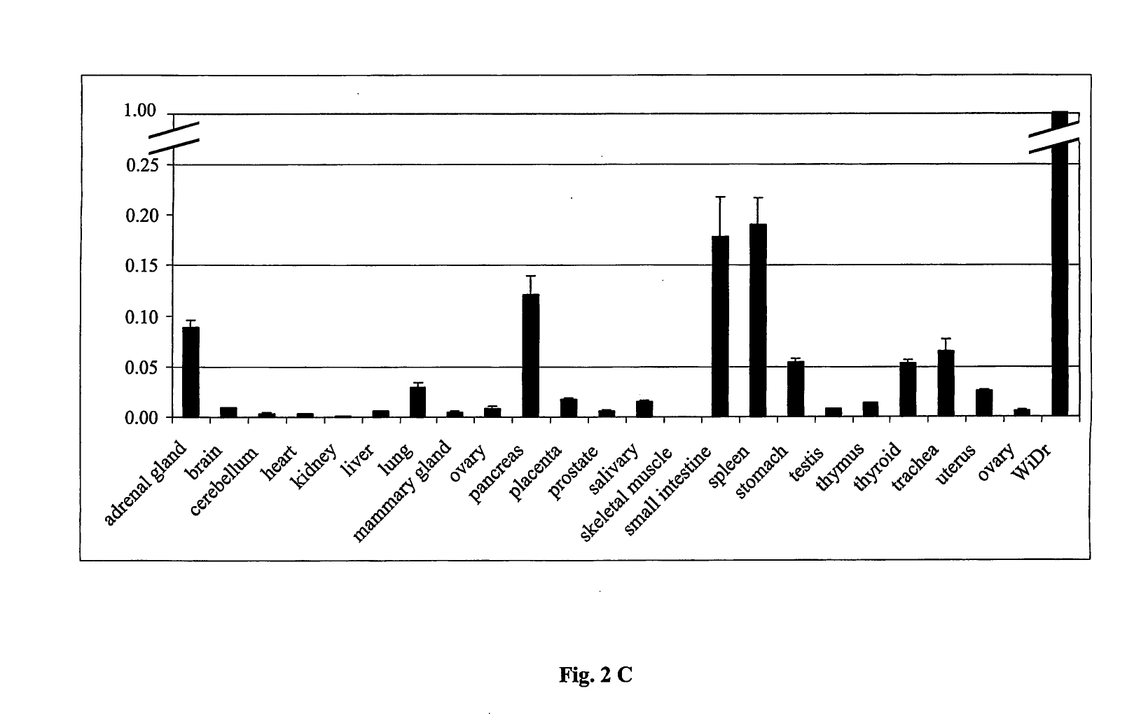

Sap-1 mRNA expression was examined in primary cells and various cancer cell lines, using a multiple tissue poly(A)RNA dot-blot, and also using quantitative PCR (TaqMan) on a set of cDNA samples from human tissues (FIG. 2). FIG. 2A shows that Sap-1 mRNA is overexpressed in Lung carcinoma and colon adenocarcinoma cell lines (lanes 2, 3, 5 and 6), but not in primary endothelial cells (lane 1), suggesting that Sap-1 may have a role in carcinogenesis of lung and colon carcinoma. In normal tissues (FIG. 2B, dot-blot) the highest expression is in various parts of the gastrointestinal tract, adrenal gland and spleen, plus weak CNS expression. This expression pattern is confirmed in the TaqMan data with highest expression in spleen, intestine and adrenal gland. Overall expression is very low with respect to a large panel of other phosphatases (data not shown). Sap-1 mRNA expression was also analyzed in RNA from colon adenocarcinoma WiDr cells. As shown in FIG. 2C, these cells express over 5-fold more Sap-1 than intestinal tissue. Overall, these data confirm earlier findings that Sap-1 is specifically overexpressed in gastrointestinal cancers (Seo et al. 1997).

### Example 3

### Sap-1 has Substrate Specificity for src-Family Kinases

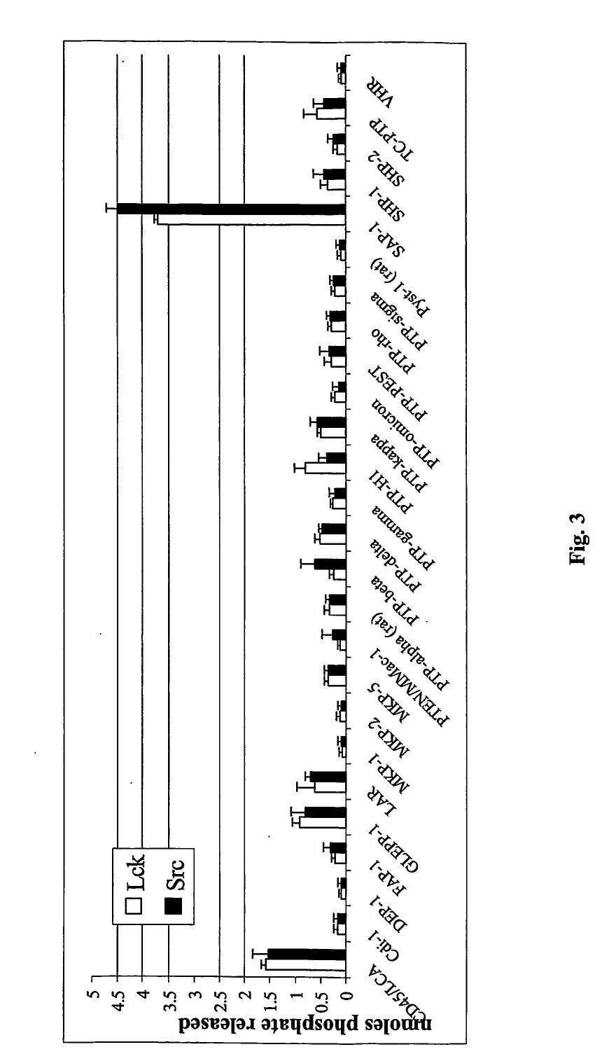

As a first step in understanding how overexpression of Sap-1 is related to cancer the enzyme's capacity to dephosphorylate phosphopeptides that correspond to the src- or lck-derived C-terminal autoinhibitory sequence was investigated. Catalytic domains (˜250 amino acids) of a large number of PTPs were prepared and incubated with the phosphopeptides. As shown in FIG. 3, Sap-1 has relative high phosphatase activity in this assay, with a measured Km of 23 μM (not shown). Another PTP with good selectivity for lck is CD45. This observation fits well with an earlier finding that CD45 inhibits lck activity (D'Oro and Ashwell 1999). The observed selectivity pattern seen for the lck and src peptides is dramatically different from selectivity for other peptides that were tested (data not shown. Additional domains of both the PTPs and their targets are likely to contribute to substrate selectivity. Nevertheless it is clear that Sap-1 can potentially dephosphorylate src at this critical phosphotyrosine.

Recognition and Activation of c-src by Sap-1

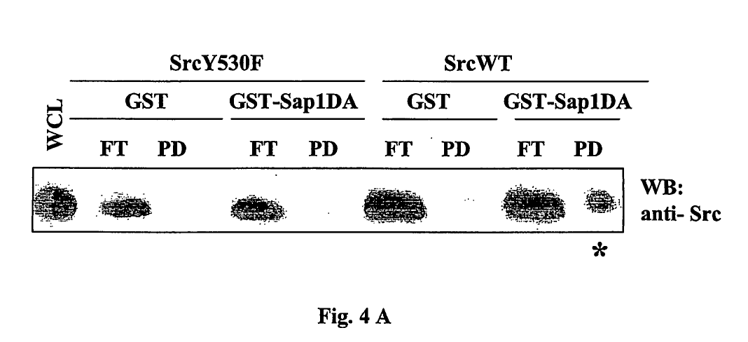

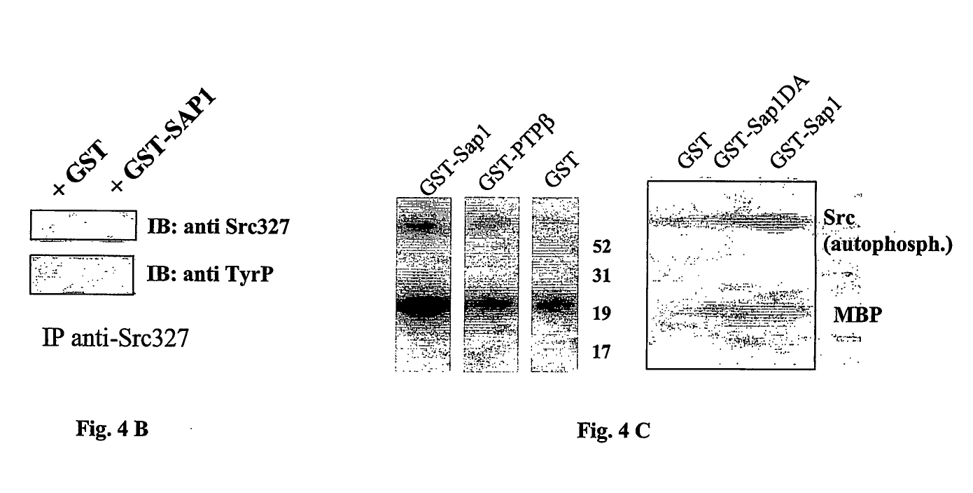

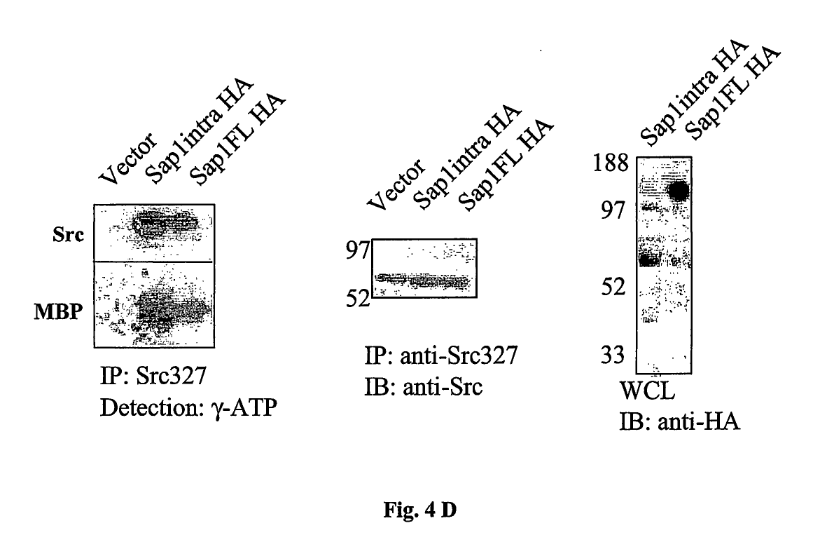

In order to see if Sap-1 interacts with activated with c-src, src was overexpressed in COS cells. In this system, >90% of src is phosphorylated at Tyr₅₃₀ and inactive (Bjorge et al. 2000a). As a control, a Y530F mutant was used that is oncogenic and undergoes Tyr₄₁₉ phosphorylation. In order to study recognition of src by Sap-1 a PTP trapping mutant Sap-1 DA was used in which the aspartic acid at position 986 is mutated into an alanine. Such mutants retain substrate specificity but remain tightly associated with their substrate without dephosphorylating it (Flint et al. 1997). As shown in FIG. 4 wild type, but not mutant src could be “pulled down” from overexpressing cells with substrate-trapping Sap-1 (FIG. 4A, last lane). Neither wild-type Sap-1 nor GST alone pulled down src. The fact that Sap-1 DA was unable to interact with Y530F-src suggests that it has no affinity for phosphorylated Tyr₄₁₉.

It was further tested whether Sap-1 can dephosphorylate full-length src. Src was immunoprecipated from overexpressing 3T3 cells and incubated for 5 min with GST or GST-Sap-1 (wild-type enzyme). After treatment with the phosphatase, src could no longer be detected with an anti-Phosphotyrosine antibody, suggesting that src had been dephosphorylated (FIG. 4B).

Next, it was analyzed whether dephosphorylation of c-src Tyr₅₃₀ by Sap-1 results in activation of the kinase. Immunoprecipated src was incubated with GST, GST-Sap-1 or GST-PTP-β, a PTP that is related to Sap-1. After stopping the reaction with sodium vanadate, a potent, nonspecific phosphatase inhibitor, each sample was incubated with radiolabelled ATP and MBP (myelin basic protein), a src kinase substrate. Finally the mixtures were run on SDS-acrylamide gels and radioactive bands visualized. As shown in FIG. 4C, treatment of src with Sap-1 resulted in an increased production of radioactive MBP, as well as src autophosphorylation (upper band). In a separate experiment (FIG. 4C, right panel) it was found that the GST-Sap1DA trapping mutant had the same effect. Presumably the Sap1 mutant binds the src phosphorylated Tyr₅₃₀ and blocks interaction of the C-terminal domain with src's SH2 domain, resulting in activation of the kinase activity.

It was subsequently checked if activation of c-src by Sap-1 occurred in vivo. Cells were co-transfected with expression vectors for c-src plus full-length or ECD (extracellular domain)-deleted Sap-1. Src was subsequently immunoprecipitated and tested for kinase activity. As shown in FIG. 4D, Sap1 co-transfection resulted in increased src kinase activity. The increase in kinase activity was larger for the Sap-1 construct that lacked the ECD, even though controls indicated it was expressed at somewhat lower levels than the Sap-1 FL (full-length) construct (FIG. 4D, right panel). This suggests that Sap-1 is inhibited by its full-length extracellular domain through the formation of homo- or heteromers.

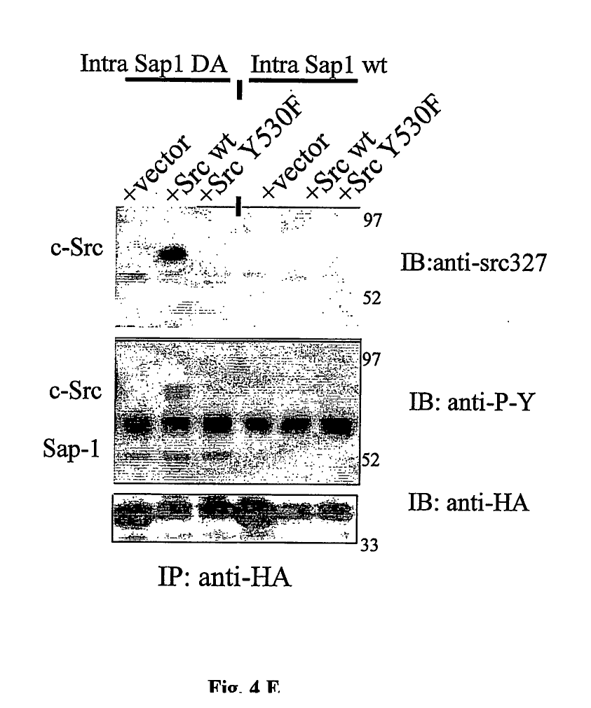

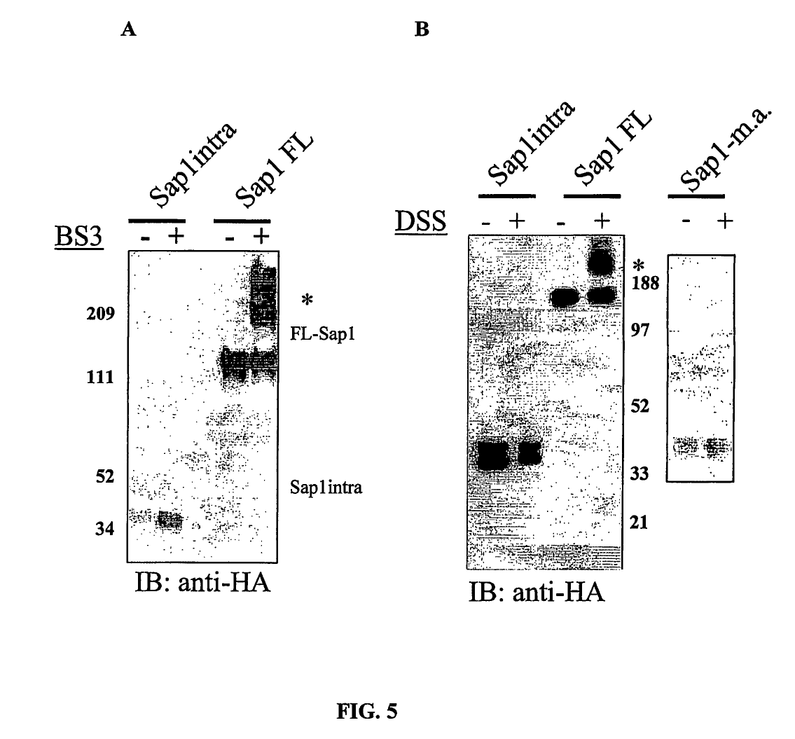

Finally the reverse experiment was performed to demonstrate in vivo interaction between c-src and Sap-1. HA-tagged Sap-1 was immunoprecipitated from cell extracts, and the presence of c-src detected on Western blot (FIG. 4E, upper panel). The substrate-trapping mutant of Sap-1 (lanes 1-3) but not wild-type Sap-1 (lanes 4-6) was able to immunoprecipitate c-src. Again, the Y530F c-src mutant showed much weaker interaction (lane 3). The phosphorylation state of proteins in the extracts was also examined (FIG. 5E, middle panel). As expected, c-src is autophosphorylated, but also Sap-1. The phosphorylation state of the latter appears independent of c-src overexpression. Wild-type Sap-1 is not phosphorylated (lanes 4-6). The band between src and Sap on this blot is the anti-HA antibody.

### Example 4

### The Sap-1 Extracellular Domain Engages in Multimerization

If full-length Sap-1 ECDs forms stable dimers they may be chemically cross-linked by treating cells with suitable compounds. COS7 cells were transiently transfected with full-length or ECD-deleted forms of Sap-1, followed by treatment with BS3 (Bis-Sulfosuccinimidyl-Suberate), a chemical cross-linker. The cell lysates were subsequently analyzed on Western blot using an anti-HA tag antibody. As shown in FIG. 5A, treatment of FL-Sap-1, but not the ECD-deleted form induces the formation of a complex with high MW. BS3 is not cell permeable so it is not too surprising no effect is seen on the ECD-deleted Sap-1 mutant. Therefore, transfected cells were also treated with the non-sulfonated variant, DSS (Di-Succinimidyl-Suberate), which is cell permeable. As shown in FIG. 5B, this compound was able to cross-link FL, but not ECD-deleted Sap-1. Finally, a ECD-deleted construct was tested that also lacked Sap-1's transmembrane domain but is attached to the inner cell membrane through an lck-derived myristoylation peptide (FIG. 1B). This construct could not be cross-linked (FIG. 5B, right panel). These results indicate that full-length Sap-1 is at least partly multimerized, and that the ECD is essential for forming multimers.

Involvement of Cysteine(s) in Multimerization

The corrected Sap-1 extracellular sequence (FIG. 1A) has an odd number (13) of cysteins. Since the extracellular environment is normally non-reducing, a cysteine in this domain might be available for intramolecular disulphur bridges. Therefore, extracts from cells that overexpress Sap-1 were isolated in presence of iodoacetamide, which blocks free sulhydryl groups, in order to block the formation of disulfide bridges during the lysis process. The samples were then run on polyacrylamide gels under non-reducing and reducing conditions, and Sap-1 was detected on Western blot using the anti-HA tag antibody. As shown in FIG. 6A, high-MW Sap-1 could be seen under non-reducing migration conditions, but not for the ECD-deleted form of Sap-1. It was next tested whether treating the cells with reducing agent Dithiothreitol (DTT) could disrupt the multimers. As shown in FIG. 6B, incubating Sap-1 overexpressing cells for 30 minutes in DTT resulted in a dose-dependent decrease of multimerized Sap-1 as detected on non-reducing gel. This DTT-induced disruption was reversible. Treating cells for 30 minutes at 50 mM DTT, followed by a washout and 15 minutes recovery in absence of DTT fully restored formation of the multimers (FIG. 6B, last two lanes).

Sap-1 Forms Dimers

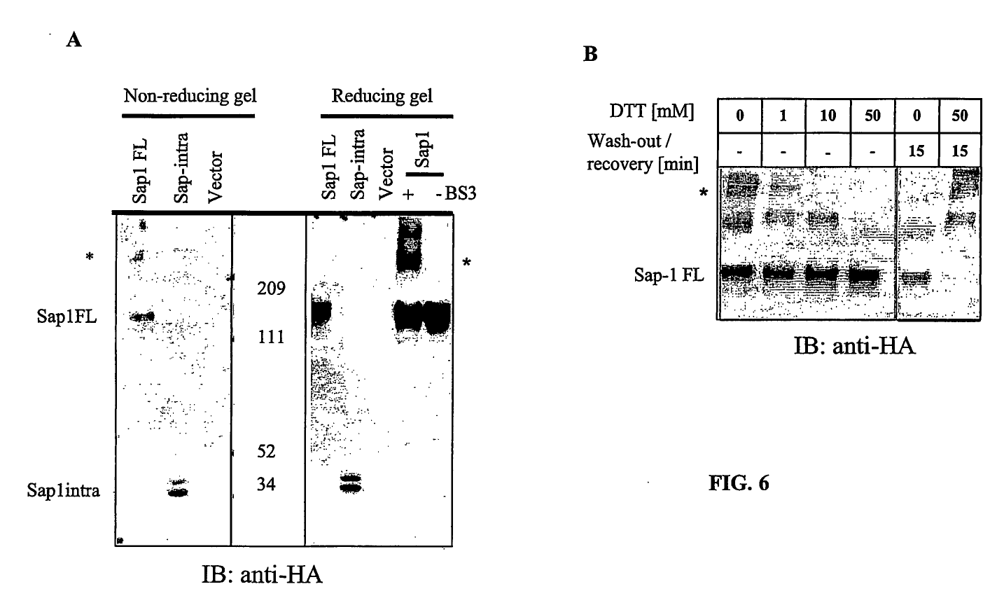

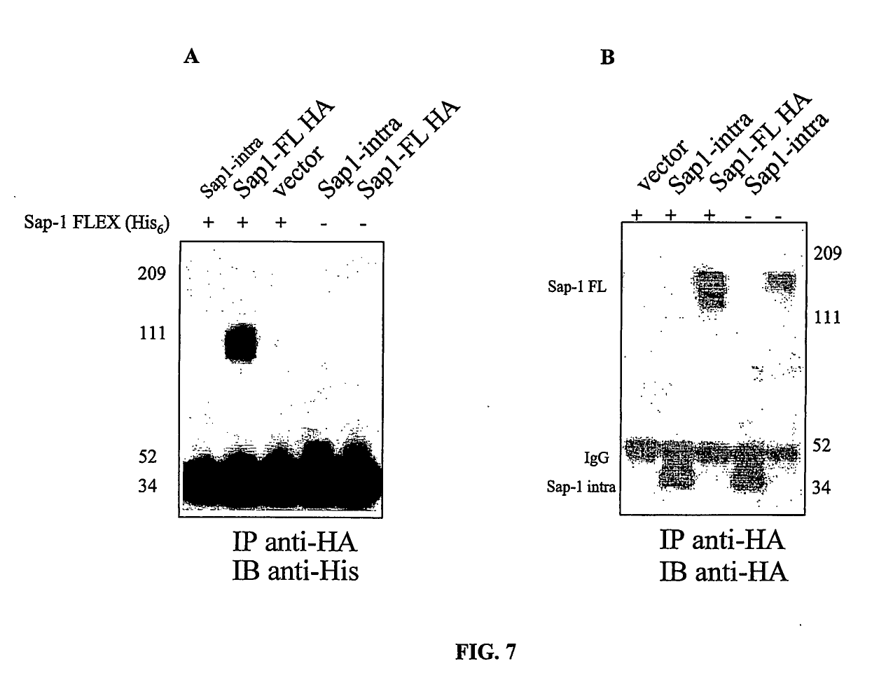

Although the size of the Sap-1 multimers shown in FIGS. 5 and 6 suggests that Sap-1 forms homo-dimers it is also possible that it interacts with other proteins. In order to address this question, Sap-1 constructs with different antigenic tags were overexpressed. An ECD-containing, His₆/Myc-tagged Sap-1 construct was co-expressed together with various other tagged Sap-1 constructs. Proteins were immunoprecipitated using an anti-HA antibody and co-immunoprecipitation of HA-tagged Sap-1 proteins were detected by Western blot, using an anti-HA antiserum. As shown in FIG. 7, HA-tagged full length, but not ECD-deleted Sap-1 could interact with the His₆-tagged FL Sap-1 construct.

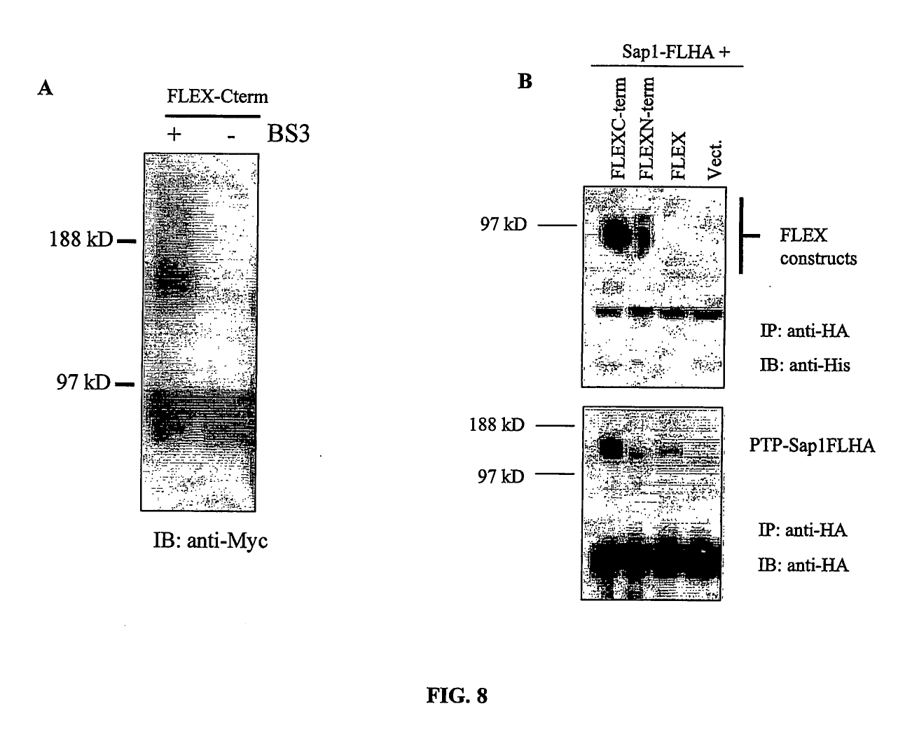

The Sap-1 extracellular domain has eight fibronectin (FN) III-like domains. In order to investigate which part of the receptor is sufficient for dimer formation myc-tagged Sap-1 constructs were generated that lacked either the three N-terminal repeats (“FLEX-Cterm”) or the two C-terminal ones (“FLEX-Nterm”; see FIG. 1B). FLEX-Cterm could still be efficiently cross-linked with BS3 (FIG. 8A). Both constructs, when co-transfected with HA-tagged FL-Sap-1, were efficiently co-immunoprecipitated with anti-HA antiserum, followed by immunodetection with anti-myc (FIG. 8B). This suggests redundancy in the N- and C-terminal domains, or that dimerization is mediated through the central domain with three FNIII-like repeats. The binding domain may be located in FNIII-like repeats 5 and 6. It is also possible that interaction occurs through multiple domains.

### Example 5

### Sap-1 Dimerization is Associated with Enzymatic Activity

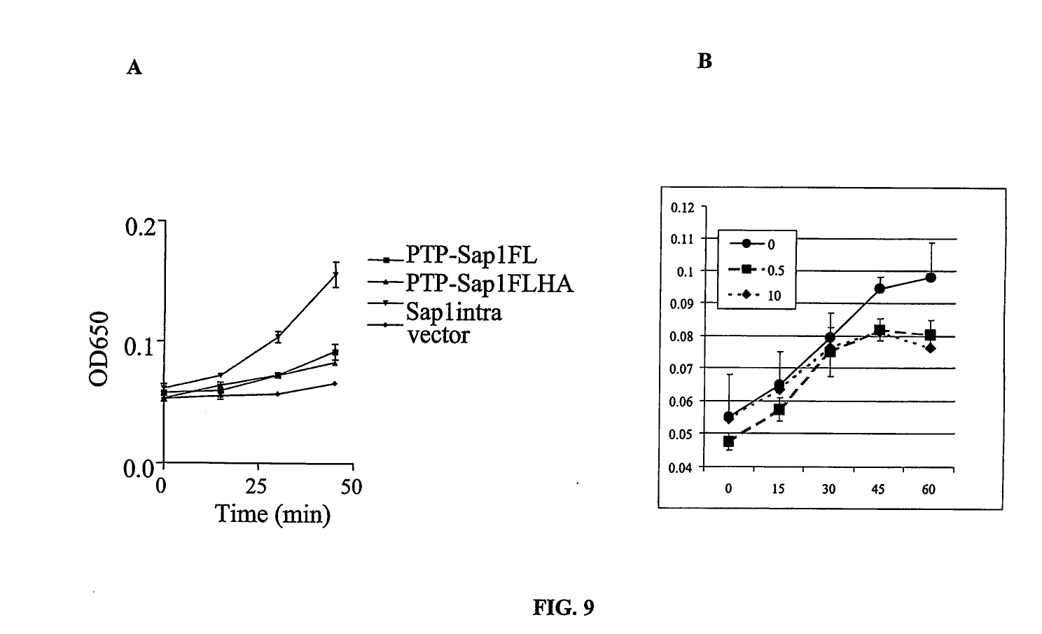

As shown above, an ECD-deleted Sap-1 construct was more efficient than the full-length version in activating c-src (FIG. 4D). It has further been shown above that the ECD mediates Sap-1 dimerization, suggesting that Sap-1 dimerization results in inhibition of Sap-1's catalytic activity. In order to address this more directly, membrane fractions were prepared from cells that expressed FL and ECD-deleted versions of Sap-1, and their activity was tested. Non-transfected cell membrane preparations have only very low PTP activity. As shown in FIG. 9A, preparations with the ECD-deleted form have higher catalytic activity than those expressing FL-Sap1. The membrane fractions were also tested in presence of a cross-linking antibody. As shown in FIG. 9B, addition of a monoclonal anti-HA antiserum results in a dose-dependent decrease of Sap-1 enzymatic activity, which can be ascribed to the effect of the antiserum's cross-linking ability.

### Example 6

### Inhibition of Sap-1 by Antisense Approach

Methods

Streptolysin Transfection and Proliferation Assay:

Cells were distributed in triplicate into 96 well plate at different dilution (1000-10000 cells/well). The next day, cells were transfected: they were first washed twice with PBS 1× and the buffer (137 mM NaCl, 10 mM PIPES pH 7.4, 5.6 mM glucose, 2.7 mM KCl, 2.7 mM EGTA, 0.1% BSA and 1 mM Na-ATP) containing streptolysin-O (0.4 U final) and oligodeoxynucleotides, ODN, (1 mM final) was incubated for not more than five minutes on cells. Afterwards, 200 μl of complete medium were added to each well for 15 to 40 hours. BrdU (Roche) was then added to the cell and the manufacturer's instructions were followed. We have performed several control, which are +/− streptolysin-O, +/− ODN and +/− BrdU.

The sequences of the primers were (AS: antisense, S: scramble): AS1: CCA GCC ATG CCT CCA GAC ACT, S1: TGC CCA CAC TCA AGC ACC CTG, AS2: TGA CCC GGG TCC AAG GCC AT, S2: GCG CGC TAG CCA CTT CGG AA, AS3: TGG TGT CTG TTG TGT TTC GA, S3: AGG GTC GGT TTT TTG GTT CT.

Colony Formation Assay:

3T3 cells (2×10⁵) cells were transfected with 2 μg of DNA (90% of the plasmid containing the gene of interest and 10% of pCIE-EGFP in order to detect the efficiency of transfection). After 24 hours, cells were harvested and 1/10 was poor in 6-well plate format (the rest was lysed in RIPA for western analysis), in presence of 3% serum and G418 (500 μg/ml). The medium was changed every three days and after 2 weeks, cells were colored with trypane blue and colonies were counted.

RNAi Design and Transfection:

RNAi were designed following the instructions given by Dharmacon: the template was chosen 75 bases downstream from the start codon where the first M dimer is present. Then the following 19 nucleotides are recorded and tested against several criteria: GC content between 30-70%, avoid primer-dimer formation and no sequences must be recognized among the cDNA database (NCBI), except the one of interest. We have ordered dimerized RNA, ready to be used.

Transfection was executed using oligofectamine reagent (Life Technologies) for WiDr cells and TranslT-TKO (Mirus) for COS7 cells. For both type of reagents, we have used in 6 well format: 60 pmole of siRNA duplex and 8 μl of TranslT-TKO or 3 μL of oligofectamine. Every experiment was done by cotransfecting pCIE-EGFP plasmid. Cells were then left 24 (COS7) to 48 (WiDr) hours and used for further experiments.

Results

A synthetic antisense-based approach was used in order to assess blockade of Sap-1 activity within the cell. First, oligo-deoxynucleotides (ODN) were used combined with streptolysin (a bacterial toxin creating membrane pores) (Barry et al., 1993), and second interfering RNA (RNAi) (Tuschl et al., 1999) were applied.

Transfection with oligodeoxynucleotides was performed using Steptolysin-O (Barry et al., 1993). Three different antisense oligos (AS1-3) and their cognate sense (S1-3) nucleotides were designed on the basis of the mRNA sequence of PTP-SAP1. The exposed loops as predicted from the mRNA's two-dimensional structure were targeted in order to increase the efficiency of binding (data not shown). The read out of the experiment was the proliferation of WiDr cells, that was tested by adding a nucleotide mimetic, BrdU. AS2 seemed to be the most efficient on this assay, but only a 20% of reduction of the proliferation was observed (results not shown).

A novel form of antisense, the RNAi (Tuschl et al., 1999) was also tested. In order to test the efficiency of the synthetic RNAis, their ability to reduce PTP-SAP1 (or, as a control, PTP-β) expression in transfected COS7 cells was tested. Interestingly, RNAi-9 did interfere with PTP-β demonstrating that contrarily to what was previously published (Tuschl et), unspecific recognition of RNAi happens. The second step was to test them in their intended cellular background, namely WiDr cells. After transfection, RNAi efficiency was monitored by adding a plasmid encoding for enhanced green fluorescent protein, EGFP. Optimal conditions can lead to 60% of transfected cells (data not shown). After 48 hours, cells were harvested and total RNA was prepared. This RNA was further reverse transcribed and amplified by TaqMan using specific oligonucleotides for PTP-SAP1. This permits to determine the precise amount of PTP-SAP1 messenger still available. Unfortunately, the expression of PTP-SAP1 was quite low. Nevertheless, one construct gave a reproducible 1.5× reduction of PTP-SAP1 mRNA.

### CONCLUSION

In the work described in the examples above it is shown that the ECD of Sap-1 is essential for Sap-1 dimerization, that this dimerization reduces the catalytic activity of Sap-1 and that Sap-1 can activate c-src oncogenic kinase activity. Therefore, blocking Sap-1, either through inhibitors directed against its catalytic domain, or through agents that increase or stabilize dimerization may be useful in treatment or prevention of gastrointestinal or other cancers, such as lung carcinomas. It is likely that the fibronectin-III-like repeats in the ECD themselves are responsible for the dimerization, since the N- and C-terminal ECD domains are redundant for dimerization FIG. 8. Such dimerization may be accomplished with bivalent antibodies, or multimeric, fibronectin-binding compounds. For the ligation of Sap-1 ECD naturally occurring fibronectin-binding proteins have been described such as collagen (Stanton et al. 2002), tenascin-C (Huang et al. 2001), heparin (Kapila et al. 2001) and integrins (Boettiger et al. 2001). From these proteins, Sap-1 cross-linkers may be manufactured.

The chemical cross-linking experiments do not allow to determine what percentage of Sap-1 molecules are dimerized, since the efficacy of the chemical reaction is difficult to estimate. However, cells that are incubated in a (natural) non-reducing environment show at least 50% dimerization (FIG. 7B). It was also shown that cysteine bridges are important for ECD dimerization, and possibly an unpaired cysteine is available for dimerization. Alternatively, treatment with reducing agent may disrupt the normal tertiary protein structure and inhibit ECD protein-protein interaction indirectly. However, deletion of the membrane-proximal cysteine in the ECD had no effect on dimerization (data not shown).

It is at present believed that by showing that Sap-1 is capable of activating c-src a mechanism is provided that may explain why Sap-1 overexpression accompanies the malignant transformation of gastrointestinal tumors. Overexpressed Sap-1 in 3T3 cells appeared toxic to cells, with lower-than-anticipated amounts of Sap-1 expressed (not shown, and Noguchi et al. 2001)

### REFERENCES

1. Andersen, J. N., Mortensen, O. H., Peters, G. H., Drake, P. G., Iversen, L. F., Olsen, O. H., Jansen, P. G., Andersen, H. S., Tonks, N. K., and Moller, N. P. 2001. Structural and evolutionary relationships among protein tyrosine phosphatase domains. Mol. Cell. Biol. 21: 7117-7136.
2. Barry, E. L., Gesek, F. A., and Friedman, P. A. (1993) Introduction of antisense oligonucleotides into cells by permeabilization with streptolysin O. Biotechniques 15, 1016-1018, 1020
3. Bhandari, V., Lim, K. L., and Pallen, C. J. 1998. Physical and functional interactions between receptor-like protein-tyrosine phosphatase alpha and p59fyn. J Biol Chem 273: 8691-8698.
4. Bjorge, J. D., Jakymiw, A., and Fujita, D. J. 2000a. Selected glimpses into the activation and function of Src kinase. Oncogene 19: 5620-5635.
5. Bjorge, J. D., Pang, A., and Fujita, D. J. 2000b. Identification of protein-tyrosine phosphatase 1B as the major tyrosine phosphatase activity capable of dephosphorylating and activating c-Src in several human breast cancer cell lines. Journal of Biological Chemistry 275: 41439-41446.
6. Blaskovich, M. A., and Kim, H. O. 2002. Recent discovery and development of protein tyrosine phosphatase inhibitors. Expert Opin. Ther. Targets 12: 871-905.
7. Boettiger, D., Lynch, L., Blystone, S., and Huber, F. 2001. Distinct ligand-binding modes for integrin alpha(v)beta(3)-mediated adhesion to fibronectin versus vitronectin. J. Biol. Chem. 276: 31684-31690.
8. Bolen et al., 1992, FASEB J., 6:3403-3409
9. Brady-Kalnay, S. M., and Tonks, N. K. 1995. Protein tyrosine phosphatases as adhesion receptors. Curr. Opin. Cell Biol. 7: 650-657.
10. Buggemann et al., Eur. J. Immunol. 21:1323-1326 (1991)
11. Cahir McFarland, E. D., Hurley, T. R., Pingel, J. T., Sefton, B. M., Shaw, A., and Thomas, M. L. 1993. Correlation between Src family member regulation by the protein-tyrosine-phosphatase CD45 and transmembrane signaling through the T-cell receptor. Proc Natl Acad Sci USA 90: 1402-1406.
12. Cheng, A., Bal, G. S., Kennedy, B. P., and Tremblay, M. L. 2001. Attenuation of adhesion-dependent signaling and cell spreading in transformed fibroblasts lacking protein tyrosine phosphatase-1B. Journal of Biologial Chemistry 276: 25848-25855.
13. Cook, W. S., and Unger, R. H. 2002. Protein tyrosine phosphatase 1B: a potential leptin resistance factor of obesity. Dev. Cell 2: 385-387.
14. den Hertog, J., Blanchetot, C., Buist, A., Overvoorde, J., van der Sar, A., and Tertoolen, L. G. 1999. Receptor protein-tyrosine phosphatase signalling in development. Int. J. Dev. Biol. 43: 723-733.
15. den Hertog, J., Pals, C. E., Peppelenbosch, M. P., Tertoolen, L. G., de Laat, S. W., and Kruijer, W. 1993. Receptor protein tyrosine phosphatase alpha activates pp60c-src and is involved in neuronal differentiation. Embo J 12: 3789-3798.
16. D'Oro, U., and Ashwell, J. D. 1999. The CD45 tyrosine phosphatase is an inhibitor of Lck activity in thymocytes. J. Immunol. 162: 1879-1883.
17. Espanel, X., Wälchli, S., Pescini Gobert, R., El Alama, M., Curchod, M.-L., Gullu-Isler, N., and Hooft van Huijsduijnen, R. 2001. Pulling Strings below the Surface: Hormone Receptor Signaling through Inhibition of Protein Tyrosine Phosphatases. Endocrine 15: 19-28.
18. Flint, A. J., Tiganis, T., Barford, D., and Tonks, N. K. 1997. Development of “substrate-trapping” mutants to identify physiological substrates of protein tyrosine phosphatases. Proc. Natl. Acad. Sci. U.S.A. 94:1680-1685.
19. Galaktionov, K., Lee, A. K., Eckstein, J., Draetta, G., Meckler, J., Loda, M., and Beach, D. 1995. CDC25 phosphatases as potential human oncogenes. Science 269: 1575-1577.
20. Grantham (1974), Science, 185. 862-864.
21. Hooft van Huijsduijnen, R. 1998. Protein Tyrosine Phosphatases: Counting the Trees in the Forest. Gene 225: 1-8.
22. Huang, W., Chiquet-Ehrismann, R., Moyano, J. V., Garcia-Pardo, A., and Orend, G. 2001. Interference of tenascin-C with syndecan-4 binding to fibronectin blocks cell adhesion and stimulates tumor cell proliferation. Cancer Res. 61: 8586-8594.
23. Hurley, T. R., Hyman, R., and Sefton, B. M. 1993. Differential effects of expression of the CD45 tyrosine protein phosphatase on the tyrosine phosphorylation of the lck, fyn, and c-src tyrosine protein kinases. Mol Cell Biol 13: 1651-1656.
24. Kapila, Y., Doan, D., Tafolla, E., and Fletterick, R. 2001. Three-dimensional structural analysis of fibronectin heparin-binding domain mutations. J. Cell. Biochem. 81: 156-161.
25. Kiener, P. A., and Mittler, R. S. 1989. CD45-protein tyrosine phosphatase cross-linking inhibits T cell receptor CD3-mediated activation in human T cells. J. Immunol. 143: 23-28.
26. Ledbetter, J. A., Schieven, G. L., Uckun, F. M., and Imboden, J. B. 1991. CD45 cross-linking regulates phospholipase C activation and tyrosine phosphorylation of specific substrates in CD3/Ti-stimulated T cells. J. Immunol. 146: 1577-1583.
27. Liu, X., Brodeur, S. R., G., Songyang, Z., Cantley, L. C., Laudano, A. P., and Pawson, T. 1993. Regulation of c-Src tyrosine kinase activity by the Src SH2 domain. Oncogene 8: 1119-1126.
28. Majeti and Weiss, 2001, Chem. Rev. 101, 2441-2448.
29. Matozaki, T., and Kasuga, M. 1996. Roles of protein-tyrosine phosphatases in growth factor signalling. Cell. Signal. 8: 13-19.
30. Matozaki, T., Suzuki, T., Uchida, T., Inazawa, J., Ariyama, T., Matsuda, K., Horita, K., Noguchi, H., Mizuno, H., Sakamoto, C., et al. 1994. Molecular cloning of a human transmembrane-type protein tyrosine phosphatase and its expression in gastrointestinal cancers. J. Biol. Chem. 269: 2075-2081.
31. Mendez, M. M., Green, L. L., Corvalan, J. R. F., Jia X-C., Maynard-Currie, E. E., Yang, X-D., Gallo, M. L., Louie, D. M., Lee, D. V., Erickson, K. L., Luna, J., Roy, C. M-N., Abderrahim, H., Kirshenbaum, F., Noguchi, M., Smith, D. M., Fukushima, A., Hales, J. F., Finer; M. H., Davis, C. G., Zsebo, K. M. and Jakobovits, A. (1997). “Functional transplant of megabase human immunoglobulin loci recapitulates human antibody response in mice”. Nature Genetics, 15, 146-56.
32. Meng, K., Rodriguez-Pena, A., Dimitrov, T., Chen, W., Yamin, M., Noda, M., and Deuel, T. F. 2000. Pleiotrophin signals increased tyrosine phosphorylation of b-catenin through inactivation of the intrinsic catalytic activity of the receptor-type protein tyrosine phosphatase b/z. Proc. Natl. Acad. Sci. U.S.A, 97: 2603-2608.
33. Moller, N. P., Moller, K. B., Lammers, R., Kharitonenkov, A., Sures, I., and Ullrich, A. 1994. Src kinase associates with a member of a distinct subfamily of protein-tyrosine phosphatases containing an ezrin-like domain. Proceedings of the National Academy of Sciences of the United States of America 91: 7477-7481.
34. Noguchi, T., Tsuda, M., Takeda, H., Takada, T., Inagaki, K., Yamao, T., Fukunaga, K., Matozaki, T., and Kasuga, M. 2001. Inhibition of cell growth and spreading by stomach cancer-associated protein-tyrosine phosphatase-1 (Sap-1) through dephosphorylation of p130cas. J. Biol. Chem. 276: 15216-15224.
35. Pathak, M. K., and Yi, T. 2001. Sodium stibogluconate is a potent inhibitor of protein tyrosine phosphatases and augments cytokine responses in hemopoietic cell lines. J. Immunol. 167: 3391-3397.
36. Peng, Z. Y., and Cartwright, C. A. 1995. Regulation of the Src tyrosine kinase and Syp tyrosine phosphatase by their cellular association. Oncogene 11: 1955-1962.
37. Ruivenkamp, C. A., Van Wezel, T., Zanon, C., Stassen, A. P., Vicek, C., Csikos, T., Klous, A. M., Tripodis, N., Perrakis, A., Boerrigter, L., et al. 2002. Ptprj is a candidate for the mouse colon-cancer susceptibility locus Scc1 and is frequently deleted in human cancers. Nat Genet. 31: 295-300.
38. Saha, S., Bardelli, A., Buckhaults, P., Velculescu, V. E., Rago, C., St Croix, B., Romans, K. E., Choti, M. A., Lengauer, C., Kinzier, K. W., et al. 2001. A phosphatase associated with metastasis of colorectal cancer. Science 294: 1343-1346.
39. Sambrook, J., Fritsch, E. F., and Maniatis, T. 1989. Molecular cloning: a laboratory manual, 2nd edition. Cold Spring Harbor Laboratory Press, Cold Spring Harbor, N.Y.
40. Schmidt, A., Rutledge, S. J., Endo, N., Opas, E. E., Tanaka, H., Wesolowski, G., Leu, C. T., Huang, Z., Ramachandaran, C., Rodan, S. B., et al. 1996. Protein-tyrosine phosphatase activity regulates osteoclast formation and function: inhibition by alendronate. Proc. Natl. Acad. Sci. U.S.A. 93: 3068-3073.
41. Seo, Y., Matozaki, T., Tsuda, M., Hayashi, Y., Itoh, H., and Kasuga, M. 1997. Overexpression of Sap-1, a transmembrane-type protein tyrosine phosphatase, in human colorectal cancers. BioChem. BioPhys. Res. Commun. 231: 705-711.
42. Stanton, H., Ung, L., and Fosang, A. J. 2002. The 45 kDa collagen-binding fragment of fibronectin induces matrix metalloproteinase-13 synthesis by chondrocytes and aggrecan degradation by aggrecanases. BioChem. J. 364: 181-190.
43. Suhr, S. M., Pamula, S., Baylink, D. J., and Lau, K. H. 2001. Antisense oligodeoxynucleotide evidence that a unique osteoclastic protein-tyrosine phosphatase is essential for osteoclastic resorption. J. Bone Miner. Res. 16: 1795-1803.
44. Tuschl, T., Zamore, P. D., Lehmann, R., Bartel, D. P., and Sharp, P. A. (1999) Targeted mRNA degradation by double-stranded RNA in vitro. Genes Dev 13, 3191-3197
45. Tomizuka et al., Proc. Natl. Acad. Sci. USA 97:722-727 (2000)
46. Thomas, S. M., and Brugge, J. S. 1997. Cellular functions regulated by Src family kinases. Annu. Rev. Cell Dev. Biol. 13: 513-609.
47. Udaykumar, Epstein, J. S., and Hewlett, I. K. 1993. A novel method employing UNG to avoid carryover contamination in RNA-PCR. Nucleic Acids Res. 21: 3917-3918.
48. Wälchli, S., Curchod, M.-L., Pescini Gobert, R., Arkinstall, S., and Hooft van Huijsduijnen, R. 2000. Identification of tyrosine phosphatases that dephosphorylate the insulin receptor: a brute-force approach based on “substrate-trapping” mutants. J. Biol. Chem. 275: 9792-9796.
49. Wiener, J. R., Hurteau, J. A., Kerns, B. J., Whitaker, R. S., Conaway, M. R., Berchuck, A., and Bast, R. C., Jr. 1994. Overexpression of the tyrosine phosphatase PTP1B is associated with human ovarian carcinomas. Am. J. Obstet Gynecol. 170: 1177-1183.
50. Zheng, X. M., Wang, Y., and Pallen, C. J. 1992. Cell transformation and activation of pp60c-src by overexpression of a protein tyrosine phosphatase. Nature 359: 336-339.
51. Zondag, G. C., Koningstein, G. M., Jiang, Y. P., Sap, J., Moolenaar, W. H., and Gebbink, M. F. 1995. Homophilic interactions mediated by receptor tyrosine phosphatases mu and kappa. A critical role for the novel extracellular MAM domain. J. Biol. Chem. 270: 14247-14250.
52. Wahl et al., J. Nucl. Med. 24:316-325 (1983)

1.-15. (canceled)

16. A method to treat or prevent cancer comprising administering to an individual a cross-linking agent capable of cross-linking at least two molecules of the protein tyrosine phosphatase Sap-1.

17. The method of claim 16, wherein the cancer is a src-associated cancer.

18. The method of claim 16, wherein the cancer is a gastrointestinal cancer.

19. The method of claim 18, wherein the gastrointestinal cancer is selected from the group consisting of esophageal tumor, stomach cancer, small-bowel tumor, large-bowel tumor, and pancreatic cancer.

20. The method of claim 16, wherein the cross-linking agent is a proteinaceous cross-linker.

21. The method of claim 20, wherein the proteinaceous cross-linker is an antibody directed against the extra-cellular domain of Sap-1.

22. The method of claim 21, wherein the antibody is directed against a Fibronectin-type III like domain of Sap-1.

23. The method of claims 20, wherein the cross-linking agent is a monoclonal antibody.

24. The method of claims 21, wherein the cross-linking agent is a monoclonal antibody.

25. The method of claim 20, wherein the cross-linking agent is a humanized antibody.

26. The method of claims 21, wherein the cross-linking agent is a humanized antibody.

27. The method of claim 20, wherein the cross-linking agent is a human antibody.

28. The method of claims 21, wherein the cross-linking agent is a human antibody.

29. The method of claim 20, wherein the cross-linking agent is a soluble fragment of the extracellular domain of Sap-1.

30. The method of claim 20, wherein the cross-linking agent comprises one, two, three, four, five, six, seven or eight Fibronectin-type III like repeats of Sap-1.

31. The method of claim 20, wherein the cross-linking agent is selected from the group consisting of: a mutein of the proteinaceous cross-linking agent, a fused protein of the proteinaceous cross-linking agent, a functional derivative of the proteinaceous cross-linking agent, an active fraction of the proteinaceous cross-linking agent, and salt of the proteinaceous cross-linking agent.

32. The method of claim 20, wherein the cross-linking agent is a functional derivative of the proteinaceous cross-linking agent comprising at least one moiety attached to one or more functional groups, which occur as one or more side chains on the amino acid residues.

33. The method of claim 32, wherein the moiety is a polyethylene moiety.


SEQUENCE LISTING (34 sequences)

SEQ ID NO: 1 - protein, 1115 aa, Homo sapiens
MAGAGGGLGVWGNLVLLGLCSWTGARAPAPNPGRNLTVETQTTSSISLSWEVPDGLDSQNSNYWVQCTGDGGTTETRNTTATNVTVDGLGPGSLYTCSVWVEKDGVNSSVGTVTTATAPNPVRNLRVEAQTNSSIALTWEVPDGPDPQNSTYGVEYTGDGGRAGTRSTAHTNITVDGLEPGCLYAFSMWVGKNGINSSRETRNATTAHNPVRNLRVEAQTTSSISLSWEVPDGTDPQNSTYCIQCTGDGGRTETRNTTDTRVTVDGLGPGSLYTCSVWVEKDGVNSSVEIVTSTTAPNPVRNLTVEAQTNSSIALTWEVPDGPDPQNSTYGVEYTGDGGRAGTRSTAHTNITVDRLEPGCLYVFSVWVGKNGINSSRETRNATTAPNPVRNLHMETQTNSSIALCWEVPDGPYPQDYTYWVEYTGDGGGTETRNTTNTSVTAERLEPGTLYTFSVWAEKNGARGSRQNVSISTVPNAVTSLSKQDWTNSTIALRWTAPQGPGQSSYSYWVSWVREGMTDPRTQSTSGTDITLKELEAGSLYHLTVWAERNEVRGYNSTLTAATAPNEVTDLQNETQTKNSVMLWWKAPGDPHSQLYVYWVQWASKGHPRRGQDPQANWVNQTSRTNETWYKVEALEPGTLYNFTVWAERNDVASSTQSLCASTYPDTVTITSCVSTSAGYGVNLIWSCPQGGYEAFELEVGGQRGSQDRSSCGEAVSVLGLGPARSYPATITTIWDGMKVVSHSVVCHTESAGVIAGAFVGILLFLILVGLLIFFLKRRNKKKQQKPELRDLVFSSPGDIPAEDFADHVRKNERDSNCGFADEYQQLSLVGHSQSQMVASASENNAKNRYRNVLPYDWSRVPLKPIHEEPGSDYINASFMPGLWSPQEFIATQGPLPQTVGDFWRLVWEQQSHTLVMLTNCMEAGRVKCEHYWPLDSQPCTHGHLRVTLVGEEVMENWTVRELLLLQVEEQKTLSVRQFHYQAWPDHGVPSSPDTLLAFWRMLRQWLDQTMEGGPPIVHCSAGVGRTGTLIALDVLLRQLQSEGLLGPFSFVRKMRESRPLMVQTEAQYVFLHQCILRFLQQSAQAPAEKEVPYEDVENLIYENVAAIQAHKLEV

SEQ ID NO: 2 - DNA, 20 bp, Artificial Sequence
  Primer
ccagctcaccatggatgatg

SEQ ID NO: 3 - DNA, 22 bp, Artificial Sequence
  Primer
ccttaatgtcacgcacgatttc

SEQ ID NO: 4 - DNA, 20 bp, Artificial Sequence
  Primer
catgctgaccaactgcatgg

SEQ ID NO: 5 - DNA, 20 bp, Artificial Sequence
  Primer
gcgagtccagaggccagtaa

SEQ ID NO: 6 - DNA, 20 bp, Artificial Sequence
  Primer
gcgagtccagaggccagtaa

SEQ ID NO: 7 - DNA, 20 bp, Artificial Sequence
  Primer
catgctgaccaactgcatgg

SEQ ID NO: 8 - DNA, 22 bp, Artificial Sequence
  Primer
gatgggatttccattgatgaca

SEQ ID NO: 9 - DNA, 18 bp, Artificial Sequence
  Primer
ccacccatggcaaattcc

SEQ ID NO: 10 - DNA, 21 bp, Artificial Sequence
  Primer
cctagtcccagggctttgatt

SEQ ID NO: 11 - DNA, 22 bp, Artificial Sequence
  Primer
ctgtgctcccactcctgatttc

SEQ ID NO: 12 - protein, 13 aa, Artificial Sequence
  Peptide
TSTEPQYQPGENL

SEQ ID NO: 13 - protein, 13 aa, Artificial Sequence
  Peptide
FFTATEGQYQPQP

SEQ ID NO: 14 - DNA, 47 bp, Artificial Sequence
  Primer
tacccatacgacgtcccagactacgctcacaccgagagtgcaggggt

SEQ ID NO: 15 - DNA, 49 bp, Artificial Sequence
  Primer
agcgtagtctgggacgtcgtatgggtagggggcaggcgccctggcccct

SEQ ID NO: 16 - DNA, 58 bp, Artificial Sequence
  Primer
tactcgagttaagcgtagtctgggacgtcgtatgggtagacctccaacttgtgggcct

SEQ ID NO: 17 - DNA, 48 bp, Artificial Sequence
  Primer
atgaattcagcggcccatctggctgcctctttctcaggaagaaaatca

SEQ ID NO: 18 - DNA, 71 bp, Artificial Sequence
  Primer
ataagcttaccatgggctgtggctgcagctcacacccggaagatgactggaagaggaggaataagaagaag

SEQ ID NO: 19 - DNA, 25 bp, Artificial Sequence
  Primer
ctctgtggtcagccacaccgagagt

SEQ ID NO: 20 - DNA, 25 bp, Artificial Sequence
  Primer
ctcggtgtggctgaccacagagtga

SEQ ID NO: 21 - DNA, 25 bp, Artificial Sequence
  Primer
gcctggccggctcacggcgttccct

SEQ ID NO: 22 - DNA, 20 bp, Artificial Sequence
  Primer
aacgccgtgagccggccagg

SEQ ID NO: 23 - DNA, 25 bp, Artificial Sequence
  Primer
cgagaaggaagtcccgtttgaggat

SEQ ID NO: 24 - DNA, 25 bp, Artificial Sequence
  Primer
catcctcaaacgggacttccttctc

SEQ ID NO: 25 - DNA, 25 bp, Artificial Sequence
  Primer
tgtcgaaaacctcatcttcgagaac

SEQ ID NO: 26 - DNA, 25 bp, Artificial Sequence
  Primer
cggccacgttctcgaagatgaggtt

SEQ ID NO: 27 - DNA, 23 bp, Artificial Sequence
  Primer
agttccagcccggggagaacctc

SEQ ID NO: 28 - DNA, 23 bp, Artificial Sequence
  Primer
gaggttctccccgggctggaact

SEQ ID NO: 29 - DNA, 21 bp, Artificial sequence
  Primer
ccagccatgcctccagacact

SEQ ID NO: 30 - DNA, 21 bp, Artificial sequence
  Primer
tgcccacactcaagcaccctg

SEQ ID NO: 31 - DNA, 20 bp, Artificial sequence
  Primer
tgacccgggtccaaggccat

SEQ ID NO: 32 - DNA, 20 bp, Artificial sequence
  Primer
gcgcgctagccacttcggaa

SEQ ID NO: 33 - DNA, 20 bp, Artificial sequence
  Primer
tggtgtctgttgtgtttcga

SEQ ID NO: 34 - DNA, 20 bp, Artificial sequence
  Primer
agggtcggttttttggttct